(19) United States
(12) Patent Application Publication
(10) Pub. No.: US 2020/0101077 A1
(43) Pub. Date: Apr. 2, 2020

(54) COMBINATION THERAPY

(71) Applicant: Novartis AG, Basel (CH)

(72) Inventors: HUAIXIANG HAO, Malden, MA (US); Xizhong Huang, Southborough, MA (US); Luigi Manenti, Shanghai (CN); Angela Tam, Randolph, MA (US)

(21) Appl. No.: 16/689,157

(22) Filed: Nov. 20, 2019

Related U.S. Application Data
(63) Continuation of application No. 15/328,935, filed on Jan. 25, 2017, now abandoned.

(30) Foreign Application Priority Data
Jul. 25, 2014 (CN) PCT/CN2014/083014

(51) Int. Cl. A61K 31/53; C07D 487/04; A61P 35/00; C07K 16/28; A61K 39/395

(52) U.S. Cl. CPC A61K 31/53; C07D 487/04; A61K 2039/505; C07K 16/2863; A61K 39/3955; A61P 35/00

(57) ABSTRACT

The present disclosure relates to pharmaceutical products comprising a combination of (i) a MET inhibitor which is INC280 or a pharmaceutically acceptable salt or hydrate thereof and (ii) an EGFR inhibitor which is an monoclonal antibody such as cetuximab or panitumumab, which are jointly active in the treatment of proliferative diseases, corresponding pharmaceutical formulations, uses, methods, processes, commercial packages and related embodiments.

### FIELD OF THE DISCLOSURE

The present disclosure relates to pharmaceutical combinations, e.g. products, comprising a combination of (i) a MET inhibitor or a pharmaceutically acceptable salt or hydrate thereof and (ii) an EGFR (ErbB-1) inhibitor which is a monoclonal antibody, which are jointly active in the treatment of proliferative diseases, corresponding pharmaceutical formulations, uses, methods, processes, commercial packages and related embodiments.

### BACKGROUND OF THE DISCLOSURE

Drugs that were designed to act against individual molecular targets often are not appropriate to combat diseases with more than one target as cause (multigenic diseases), such as cancer or other proliferative diseases.

In order to combat such diseases, one approach is to use single multi-target drugs—however, here it is required that the targets causally involved into manifestation of a disease are all hit by the drug considered. On the other hand, multi-target drugs may lead to undesired side effects as they may also have impact on targets not involved in the disease manifestation.

A different approach is to use a combination of drugs as multi-target drugs. In the best scenario, this may lead to a combined efficiency, e.g. synergy, thus even allowing a reduction of side effects caused by the single drugs when used alone.

Occasionally, the components (combination partners) of such drugs may impact separate targets to create a combination effect, and thus may create a combination effect going beyond what is achievable with the single compounds and/or when considering their isolated effects, respectively, either in the same pathway or separate pathways, within an individual cell or in separate cells in separate tissues. Alternatively, one component may alter the ability of another to reach its target, e.g. by inhibiting of efflux pumps or the like. Yet alternatively, the combination partners may bind to separate sites of the same target. These variants of target connectivity hamper the search for appropriate combinations by hugely increasing the possible types of interactions that might be useful for combination or not.

However, a desired cooperation, or even a synergy, using such drugs may not be found in many cases. As the number of pairwise (r=2) drug combinations increases according to the formula n!/(r!(n−r)!) with the number of agents n being tested (e.g. testing 2000 agents would already generate 1,999,000 unique pairwise combinations), an appropriate screening method allowing high efficiency is necessary.

In addition, before any combination is considered, there is a crucial requirement to identify the pathways, enzymes, metabolic states or the like that are involved causally or in a supporting way in the disease manifestation.

In many cases, it is not even known at all that a given disease is multigenic.

Therefore, the search for appropriate combinations and amounts can properly be described to correspond to finding a needle in a haystack.

The proto-oncogen cMET (MET) encodes the protein Hepatocyte Growth Factor Receptor (HGFR) which has tyrosine kinase activity and is essential for embryonic development and wound healing. Upon Hepatocyte Growth Factor (HGF) stimulation, MET induces several biological responses, leading to invasive growth. Abnormal MET activation triggers tumor growth, formation of new blood vessels (angiogenesis) and metastasis, in various types of malignancies, including cancers of the kidney, liver, stomach, breast and brain. A number of MET kinase inhibitors are known, and alternatively inhibitors of HOF-induced MET (=HGFR) activation. The biological functions of c-MET (or c-MET signaling pathway) in normal tissues and human malignancies such as cancer have been well documented (Christensen, J. G. et al., Cancer Lett. 2005, 225(1):1-26; Corso, S. et al., Trends in Mol. Med. 2005, 11(6):284-292).

A dysregulated c-Met (c-MET) pathway plays important and sometimes causative (in the case of genetic alterations) roles in tumor formation, growth, maintenance and progression (Birchmeier, C. et al., Nat. Rev. Mol. Cell. Biol. 2003, 4(12):915-925; Boccaccio, C. et al., Nat. Rev. Cancer 2006, 6(8):637-645; Christensen, J. G. et al., Cancer Lett. 2005, 225(1):1-26), HGF and/or c-Met are overexpressed in significant portions of most human cancers, and are often associated with poor clinical outcomes such as more aggressive disease, disease progression, tumor metastasis and shortened patient survival. Further, patients with high levels of HGF/c-Met proteins are more resistance to chemotherapy and radiotherapy. In addition to the abnormal HGF/c-Met expression, c-Met receptor can also be activated in cancer patients through genetic mutations (both germline and somatic) and gene amplification. Although gene amplification and mutations are the most common genetic alterations that have been reported in patients, the receptor can also be activated by deletions, truncations, gene rearrangement.

The various cancers in which c-MET is implicated include, but are not limited to: carcinomas (e.g., bladder, breast, cervical, cholangiocarcinoma, colorectal, esophageal, gastric, head and neck, kidney, liver, lung, nasopharygeal, ovarian, pancreas, prostate, thyroid); musculoskeletal sarcomas (e.g., osteosarceoma, synovial sarcoma, rhabdomyosarcoma); soft tissue sarcomas (e.g., MFH/fibrosarcorna, leiomyosarcoma, Kaposi's sarcoma); hematopoietic malignancies (e.g., multiple myeloma, lymphomas, adult T cell leukemia, acute myelogenous leukemia, chronic myeloid leukemia); and other neoplasms (e.g., glioblastomas, astrocytomas, melanoma, mesothelioma and Wilm's tumor (www.vai.org/met/; Christensen, J. G. et al., Cancer Lett. 2005, 225(1):1-26).

The notion that the activated c-MET pathway contributes to tumor formation and progression and could be a good target for effective cancer intervention has been further solidified by numerous preclinical studies (Birchmeier, C. et al., Nat. Rev. Mol. Cell Biol. 2003, 4(12):915-925; Christensen, J. G. et al., Cancer Lett. 2005, 225(1):1-26; Corso, S. et al., Trends in Mol. Med. 2005, 11(6): 284-292). For example, studies showed that the tpr-met fusion gene, overexpression of c-met and activated c-met mutations (collectively referred to herein as MET) all caused oncogenic transformation of various model cell lines and resulted in tumor formation and metastasis in mice. More importantly, significant anti-tumor (sometimes tumor regression) and anti-metastasis activities have been demonstrated in vitro and in vivo with agents that specifically impair and/or block HGF/c-MET signaling. Those agents include anti-HGF and antic-Met antibodies, HGF peptide antagonists, decoy c-Met receptor, c-Met peptide antagonists, dominant negative c-Met mutations, c-Met specific antisense oligonucleotides and ribozymes, and selective small molecule c-Met kinase inhibitors (Christensen, J. G. et al., Cancer Lett. 2005, 225(1):1-26).

In addition to the established role in cancer, abnormal HGF/MET signaling is also implicated in atherosclerosis, lung fibrosis, renal fibrosis and regeneration, liver diseases, allergic disorders, inflammatory and autoimmune disorders, cerebrovascular diseases, cardiovascular diseases, conditions associated with organ transplantation (Ma, H. et al., Atherosclerosis. 2002, 164(1):79-87; Crestani, B. et al., Lab. Invest. 2002, 82(8):1015-1022; Segura-Flores, A. A. et al., Rev. Gastroenterol. Mex. 2004, 69(4)243-250; Morishita, R. et al., Curr. Gene Ther. 2004, 4(2)199-206; Morishita, R. et al., Endocr. J. 2002, 49(3)273-284; Liu, Y., Curr. Opin. Nephrol. Hypertens. 2002, 11(1):23-30; Matsumoto, K. et al., Kidney Int. 2001, 59(6):2023-2038; Balkovetz, D. F. et al., Int. Rev. Cytol. 1999, 186:225-250; Miyazawa, T. et al., J. Cereb. Blood Flow Metab. 1998, 18(4)345-348; Koch, A. E. et al., Arthritis Rheum. 1996, 39(9):1566-1575; Futamatsu, H. et al., Circ. Res. 2005, 96(8)823-830; Eguchi, S. et al., Clin. Transplant. 1999, 13(6)536-544).

The Epidermal Growth Factor Receptor (EGFR, aka ErbB-1; HER1 in humans), is a receptor for ligands of the epidermal growth factor family. Several types of cancers are known to be dependent on EGFR over-activity or over-expression, such as lung cancer, anal cancers, glioblastoma multiforme and many other mainly epithelial cancers.

Cancer is often dependent on the genetic alteration of receptor tyrosine kinases (RTKs) e.g. by point mutation, gene amplification or chromosomal translocation which leads to uncontrolled activity of these RTKs which thus become oncogenic. Cell proliferation of cancer cells is dependent on the activity of these aberrant RTKs.

When treating the resulting proliferative diseases, often inhibitors of the oncogene. RTK involved are used. However, often, after a certain time of treatment, resistance to the drug used is observed. One mechanism of resistance can involve the target RTK, compromising binding or activity of the therapeutic agent. Another mechanism is compensatory activation of an alternative kinase that continues to drive cancer growth when the primary kinase is inhibited. A well-characterized example covering both types of mechanisms is acquired resistance to the epidermal growth factor receptor (EGFR) gefitinib and erlotinib in non-small cancer (NSCLC) carrying activating EGFR mutations (see Lynch, T. J., et al., N Engl J Med, 350: 2129-2139, 2004; or Paez, J. G., et al., Science, 304: 1497-1500, 2004). For example, MET activation can compensate for loss of EGFR activity (by inhibition) by downstream activation of signal molecules such as HER3, such as MET amplification may compensate, or its ligand hepatocyte growth factor may activate MET (see Engelman, J. A., et al., Science, 316: 1039-1043, 2007; Yano, S., et al., Cancer Res, 68: 9479-9487, 2008; and Turke, A. B., et al., Cancer Cell, 17; 77-88, 2010). It is also known that MET-dependent cancer cell lines (the proliferation of which depends on the activity of MET) can be rescued from MET inhibitors by ligand-induced EGFR activation (see Bachleitner-Hofmann, T., et al., Mol Cancer Ther, 7: 3499-3508, 2008).

WO2013/149581 discloses the combination of various cMET inhibitors with various EGFR inhibitors. It relates to pharmaceutical products comprising a combination of (i) a MET inhibitor and (ii) an EGFR inhibitor, or a pharmaceutically acceptable salt or hydrate thereof, respectively, or a prodrug thereof, which are jointly active in the treatment of proliferative diseases, corresponding pharmaceutical formulations, uses, methods, processes, commercial packages and related embodiments.

### SUMMARY OF THE DISCLOSURE

The present disclosure relates to a pharmaceutical combination comprising

(i) a MET tyrosine kinase inhibitor which is INC280 having the formula

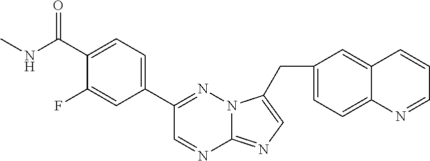 [SMILES: CNC(=O)c1ccc(-c2cnc3ncc(Cc4ccc5ncccc5c4)n3n2)cc1F]

or a pharmaceutically acceptable salt or hydrate thereof,
(ii) an EGFR tyrosine kinase inhibitor which is a monoclonal antibody.

The present disclosure also relates to a pharmaceutical combination comprising

(i) a MET tyrosine kinase inhibitor which is INC280 having the formula

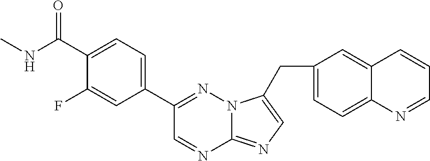 [SMILES: CNC(=O)c1ccc(-c2cnc3ncc(Cc4ccc5ncccc5c4)n3n2)cc1F]

or a pharmaceutically acceptable salt or hydrate thereof,
(ii) an EGFR tyrosine kinase inhibitor which is a monoclonal antibody, and
(iiii) at least one pharmaceutically acceptable carrier.

In one embodiment of the combination, the EGFR tyrosine kinase inhibitor is cetuximab.

In another embodiment of the combination, the EGFR tyrosine kinase inhibitor is panitumumab.

In one embodiment of the combination, the INC280 is in its dihydrochloric acid salt form.

In another embodiment, the INC280 is in the form of the dihydrochloride monohydrate salt.

In one embodiment of the combination, the MET tyrosine kinase inhibitor and the EGFR tyrosine kinase inhibitor are simultaneously, separately or sequentially administered.

The present disclosure also relates to a method of treating an EGFR tyrosine kinase activity and/or MET tyrosine kinase activity mediated disease, especially a cancer, comprising administering a pharmaceutical combination comprising

(i) a MET tyrosine kinase inhibitor which is INC280 having the formula

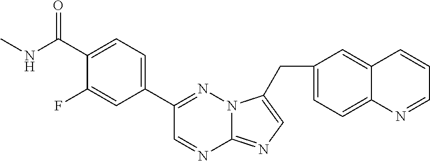 [SMILES: CNC(=O)c1ccc(-c2cnc3ncc(Cc4ccc5ncccc5c4)n3n2)cc1F]

or a pharmaceutically acceptable salt or hydrate thereof,
(ii) an EGFR tyrosine kinase inhibitor which is a monoclonal antibody, and
(iiii) optionally at least one pharmaceutically acceptable carrier.

In one embodiment of the method, the EGFR tyrosine kinase inhibitor is cetuximab.

In another embodiment of the method, the EGFR tyrosine kinase inhibitor is panitumumab.

In one embodiment of the method, the INC280 is in its dihydrochloric acid salt form.

In another embodiment, the INC280 is in the form of the dihydrochloride monohydrate salt.

In one embodiment of the method, the MET tyrosine kinase inhibitor and the EGFR tyrosine kinase inhibitor are simultaneously, separately or sequentially administered.

In one embodiment of the method, the cancer is selected from the group consisting of carcinomas (e.g., bladder, breast, cervical, cholangiocarcinoma, colorectal, esophageal, gastric, head and neck, kidney, liver, lung, nasopharygeal, ovarian, pancreas, prostate, thyroid); musculoskeletal sarcomas (e.g., osteosarcaoma, synovial sarcoma, rhabdomyosarcoma); soft tissue sarcomas (e.g., MFH/fibrosarcoma, leiomyosarcoma, Kaposi's sarcoma); hematopoietic malignancies (e.g., multiple myeloma, lymphomas, adult T cell leukemia, acute myelogenous leukemia, chronic myeloid leukemia); and other neoplasms (e.g., glioblastomas, astrocytomas, melanoma, mesothelioma and Wilm's tumor.

In one embodiment of the method, the cancer is non-small cell lung cancer (NSCLC).

In another embodiment of the method, the cancer is metastatic non-small cell lung cancer.

In another embodiment of the method, the cancer is colorectal cancer (CRC).

In another embodiment of the method, the cancer is metastatic colorectal cancer (mCRC).

In another embodiment of the method, the cancer is head and neck cancer.

In another embodiment of the method, the cancer is metastatic head and neck cancer.

In yet another embodiment of the method, the cancer is head and neck squamous cell carcinoma (HNSCC).

### DETAILED DESCRIPTION OF THE FIGURES

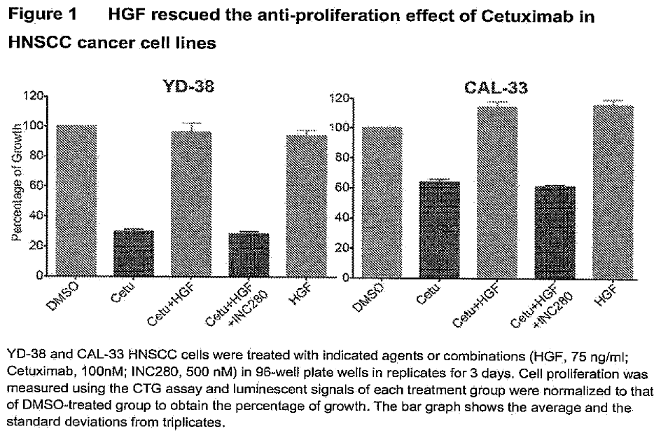

FIG. 1 illustrates that HGF rescued the anti-proliferation effect of Cetuximab in HNSCC cancer cell lines.

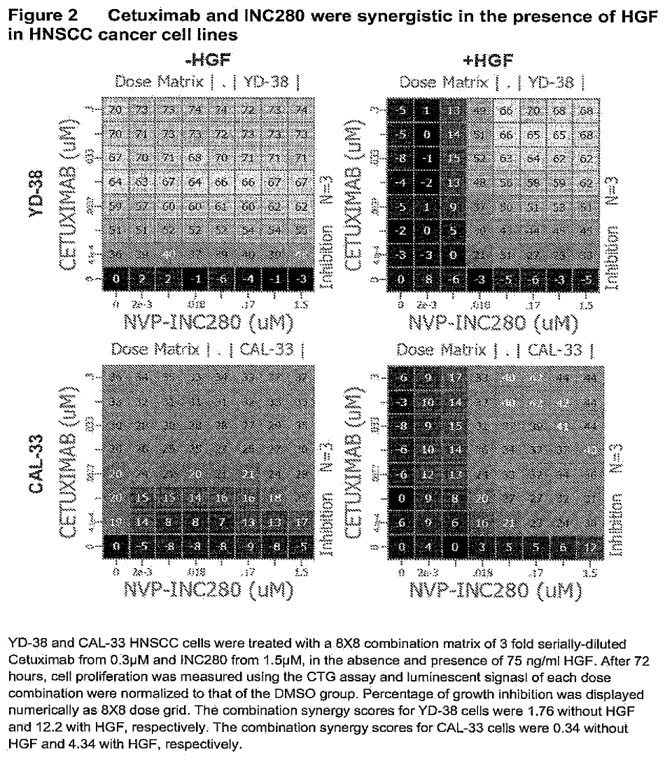

FIG. 2 illustrates that Cetuximab and INC280 were synergistic in the presence of HGF in HNSCC cancer cell lines.

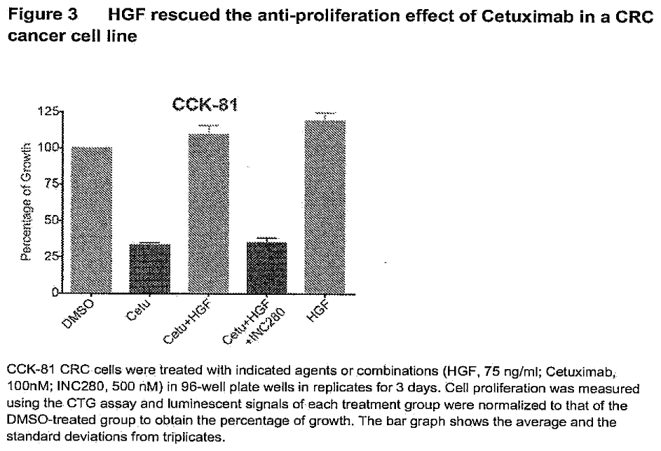

FIG. 3 illustrates that HGF rescued the anti-proliferation effect of Cetuximab in a CRC cancer cell line.

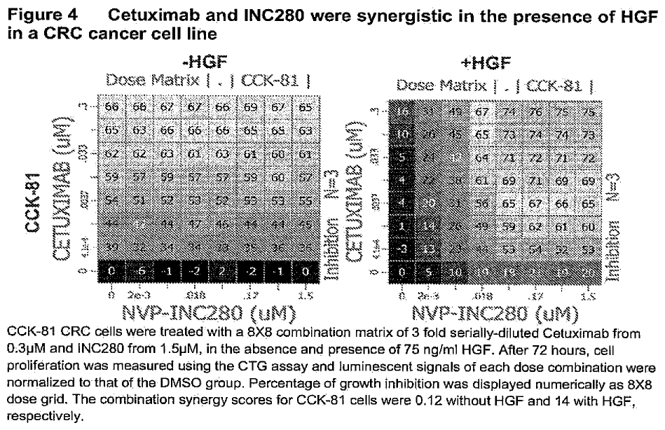

FIG. 4 illustrates that Cetuximab and INC280 were synergistic in the presence of HGF in a CRC cancer cell line.

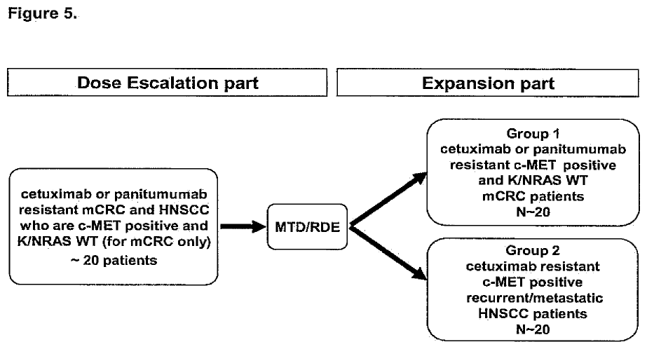

FIG. 5 illustrates how the phase Ib dose escalation part of the study will be conducted in adult c-MET positive and K/NRAS WT mCRC and c-MET positive HNSCC patients.

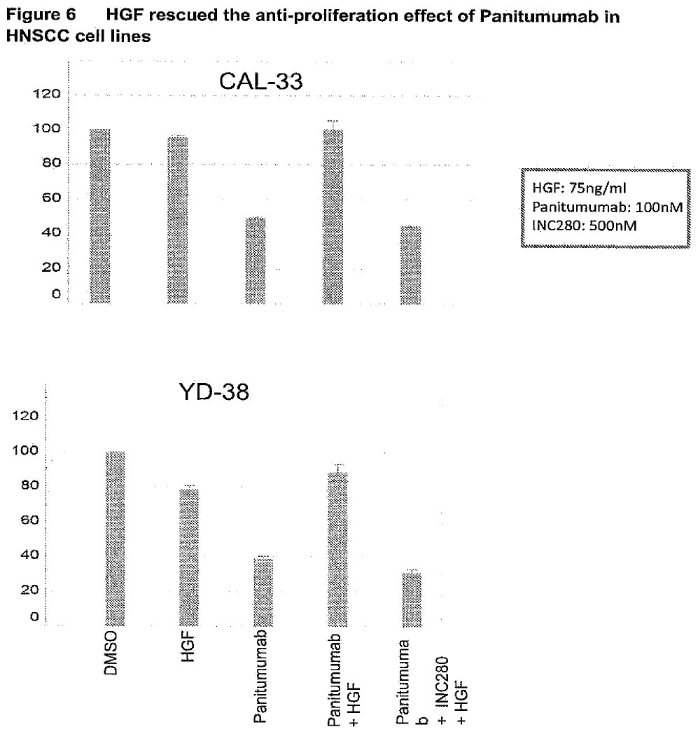

FIG. 6 illustrates that HGF rescued the anti-proliferation effect of Panitumumab in HNSCC cancer cell lines.

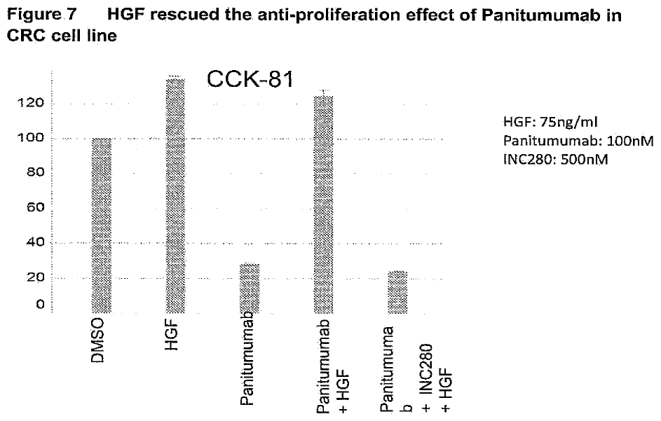

FIG. 7 illustrates that HGF rescued the anti-proliferation effect of Panitumumab in a CRC cancer cell line.

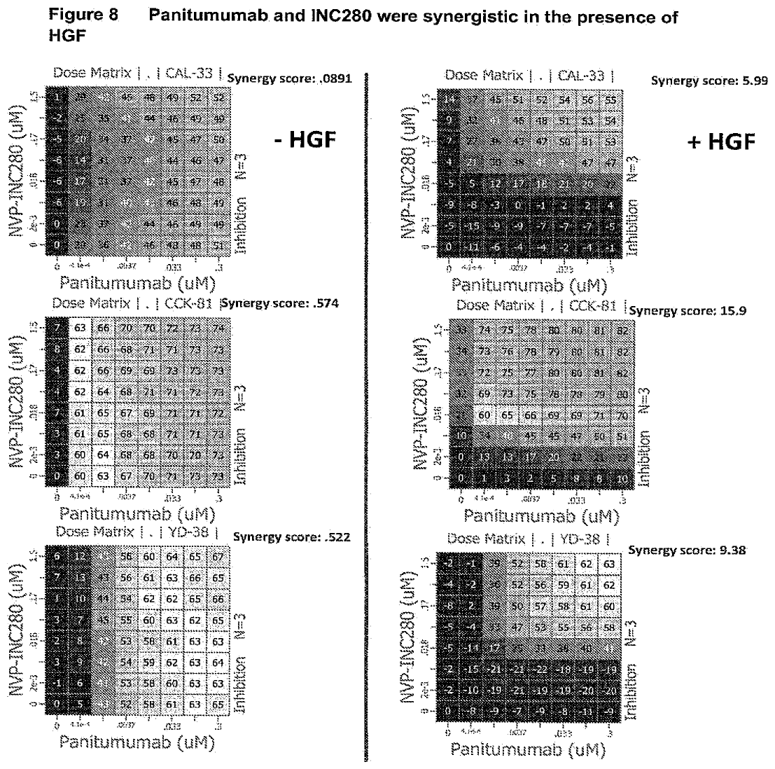

FIG. 8 illustrates that Panitumumab and INC280 were synergistic in the presence of HGF in various cancer cell lines.

### DETAILED DESCRIPTION OF THE DISCLOSURE

The present disclosure, according to a first embodiment, relates to a pharmaceutical combination (e.g. combination product) comprising (i) a MET inhibitor which is INC280 or a pharmaceutically acceptable salt and (ii) an EGFR inhibitor which is a monoclonal antibody (e.g., cetuximab or panitumumab) and at least one pharmaceutically acceptable carrier.

The present disclosure also relates to a pharmaceutical combination (e.g. combination product) comprising (i) a MET inhibitor which is INC280 or a pharmaceutically acceptable salt and (ii) an EGFR inhibitor which is a monoclonal antibody (e.g., cetuximab or panitumumab).

The chemical name of INC280 is 2-fluoro-N-methy-4-[(7-quinolin-6-yl-methyl)imidazo[1,2-b]triazin-2-yl]benzamide which has the formula

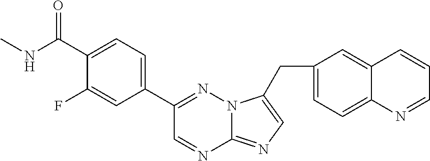 [SMILES: CNC(=O)c1ccc(-c2cnc3ncc(Cc4ccc5ncccc5c4)n3n2)cc1F]

INC280 is disclosed in WO 2008/064157, Example 7. Non-limiting examples of salt forms of INC280 are dihydrochloric acid form and dibenzenesulfonic acid salts. In particular, INC280 can be in the form of the dihydrochloride monohydrate salt (also described in U.S. Pat. No. 8,420,645). INC280 is also known by its INN which is capmatinib.

A further embodiment of this disclosure provides a combination (e.g. combination product) comprising a quantity which is jointly therapeutically effective against an EGFR tyrosine kinase activity and/or MET tyrosine kinase activity mediated disease, especially a cancer, comprising the combination partners (i) EGFR tyrosine kinase inhibitor which is a monoclonal antibody (e.g., cetuximab or panitumumab) and (ii) MET tyrosine kinase inhibitor which is INC280 or a pharmaceutically acceptable salt thereof, and optionally at least one pharmaceutically acceptable carrier material.

A further embodiment relates to the use of the inventive combination (e.g. combination product) for treating an EGFR tyrosine kinase activity and/or MET tyrosine kinase activity mediated disease, especially a cancer.

A further embodiment relates to the use of a combination of (i) an EGFR tyrosine kinase inhibitor which is a monoclonal antibody (e.g., cetuximab or panitumumab) and (ii) a MET tyrosine kinase inhibitor which is INC280 or a pharmaceutically acceptable salt thereof, for the manufacture of a medicament or a pharmaceutical product for treating an EGFR tyrosine kinase activity and/or MET tyrosine kinase activity mediated disease, especially a cancer.

There is also provided a combination comprising (i) an EGFR tyrosine kinase inhibitor which is a monoclonal antibody (e.g., cetuximab or panitumumab) and (ii) a MET tyrosine kinase inhibitor which is INC280 or a pharmaceutically acceptable salt thereof, for use in treating an EGFR tyrosine kinase activity and/or MET tyrosine kinase activity mediated disease, especially a cancer.

A further embodiment relates to a method of treating an EGFR tyrosine kinase activity and/or MET tyrosine kinase activity mediated disease, especially a cancer, with a combination of (i) an EGFR tyrosine kinase inhibitor which is a monoclonal antibody (e.g., cetuximab or panitumumab) and (ii) a MET tyrosine kinase inhibitor which is INC280 or a pharmaceutically acceptable salt thereof.

A further embodiment relates to a method for the treatment of an EGFR tyrosine kinase activity and/or MET tyrosine kinase activity mediated disease, especially a cancer, said method comprising administering an effective amount of a combination of or a combination product comprising (i) an EGFR tyrosine kinase inhibitor which is a monoclonal antibody (e.g., cetuximab or panitumumab) and (ii) a MET tyrosine kinase inhibitor which is INC280 or a pharmaceutically acceptable salt thereof to a subject in need thereof, such as a warm-blooded animal, in particular a human.

Yet a further embodiment of present disclosure relates to a pharmaceutical product or a commercial package comprising a combination product according to the disclosure described herein, in particular together with instructions for simultaneous, separate or sequential use (especially for being jointly active) thereof in the treatment of an EGFR tyrosine kinase activity and/or MET tyrosine kinase activity mediated disease, especially a cancer, in particular for use in the treatment of an EGFR tyrosine kinase activity and/or MET tyrosine kinase activity mediated disease, especially a cancer.

A further embodiment of present disclosure relates to the use of (i) an EGFR tyrosine kinase inhibitor which is a monoclonal antibody (e.g., cetuximab or panitumumab) and (ii) a MET tyrosine kinase inhibitor which is INC280 or a pharmaceutically acceptable salt thereof, for the preparation of a combination (e.g. a combination product) according to present disclosure.

The following definitions show more specific embodiments of general features or expressions which can be used to replace one, more than one or all general features or expressions in the embodiments described hereinbefore and hereinafter, thus leading to more specific embodiments.

Non-limiting examples of the EGFR tyrosine kinase inhibitor which is a monoclonal antibody includes cetuximab and panitumumab.

Cetuximab (tradename: Erbitux) is an epidermal growth factor receptor (EGFR) inhibitor used for the treatment of metastatic colorectal cancer, metastatic non-small cell lung cancer and head and neck cancer. Cetuximab is a chimeric (mouse/human) monoclonal antibody given by intravenous infusion that is manufactured and distributed in the United States by the drug companies Bristol-Myers Squibb and Eli Lily and Company and in Europe by the drug company Merck KGaA.

Panitumumab (formerly known as ABX-EGF), is a fully human monoclonal antibody specific to the epidermal growth factor receptor (also known as EGF receptor, EGFR, ErbB-1 and HER1 in humans). Panitumumab is manufactured by Amgen and marketed as Vectibix.

The cancer that is treated may be selected from the group consisting of carcinomas (e.g., bladder, breast, cervical, cholangiocarcinoma, colorectal, esophageal, gastric, head and neck, kidney, liver, lung, nasopharygeal, ovarian, pancreas, prostate, thyroid); musculoskeletal sarcomas (e.g., osteosarceoma, synovial sarcoma, rhabdomyosarcoma); soft tissue sarcomas (e.g., MFH/fibrosarcoma, leiomyosarcoma, Kaposi's sarcoma); hematopoietic malignancies (e.g., multiple myeloma, lymphomas, adult T cell leukemia, acute myelogenous leukemia, chronic myeloid leukemia); and other neoplasms (e.g., glioblastomas, astrocytomas, melanoma, mesothelioma and Wilm's tumor.

The cancer may be non-small cell lung cancer (NSCLC).

The cancer may be metastatic non-small cell lung cancer.

The cancer may be colorectal cancer (CRC).

The cancer may be is metastatic colorectal cancer (mCRC).

The cancer may be head and neck cancer.

The cancer may be metastatic head and neck cancer.

The cancer may be head and neck squamous cell carcinoma (HNSCC).

The combination of the disclosure may be particularly suitable mCRC and HNSCC patients whose tumors have become resistant to anti-EGFR treatment through activation of the MET receptor.

Compounds useful according to the disclosure can also include all isotopes of atoms occurring in the intermediates or final compounds. Isotopes include those atoms having the same atomic number but different mass numbers. Examples of isotopes that can be incorporated into compounds of the disclosure include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³²P, ³²P, ³⁵S, ³⁶Cl, ¹²⁵I respectively. Various isotopically labeled compounds of the present disclosure, for example those into which radioactive isotopes such as ³H, ¹³C, and ¹⁴C are incorporated. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT) Including drug or substrate tissue distribution assays, or in radioactive treatment of patients. In particular, an ¹⁸F or labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this disclosure can generally be prepared by carrying out the procedures disclosed in the schemes or in the examples and preparations described below by substituting a. readily available isotopically labeled reagent for a non-isotopically labeled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium, for example in the ranges given above.

Isotopically-labeled MET and/or EGFR tyrosine kinase inhibitor compounds forming part of a combination product according to the disclosure can generally be prepared by conventional techniques known to those skilled in the art or by processes analogous to those described in the accompanying Examples and Preparations using an appropriate isotopically-labeled reagents in place of the non-labeled reagent previously employed.

The present disclosure embodiments also include pharmaceutically acceptable salts of the compounds useful according to the disclosure described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

A preferred salt of INC280 is the hydrochloric acid salt, specially the dihydrochloric acid salt form.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compounds useful according to the disclosure (=being included in a combination, especially a combination product, according to the disclosure, respectively, or being used according to the disclosure, optionally also including further co-agents as defined below, that is, all active ingredients), as well as their pharmaceutically acceptable salts, can also be present as tautomers, N-oxides or solvates, e.g. hydrates. All these variants, as well as any single one thereof or combination of two or more to less than all such variants, are encompassed and to be read herein where a compound included in the inventive combination products, e.g. an EGFR tyrosine kinase inhibitor and/or a MET tyrosine kinase inhibitor, is mentioned.

The present disclosure, according to a first embodiment mentioned above and below, relates to a pharmaceutical combination, especially a pharmaceutical combination product, comprising the mentioned combination partners and at least one pharmaceutically acceptable carrier.

“Combination” refers to formulations of the separate partners with or without instructions for combined use or to combination products. The combination partners may thus be entirely separate pharmaceutical dosage forms or pharmaceutical compositions that are also sold independently of each other and where just instructions for their combined use are provided in the package equipment, e.g. leaflet or the like, or in other information e.g. provided to physicians and medical staff (e.g. oral communications, communications in writing or the like), for simultaneous or sequential use for being jointly active, especially as defined below.

“Combination product” Includes either a fixed combination in one dosage unit form, or a kit of parts for the combined administration where an EGFR tyrosine kinase inhibitor and a MET tyrosine kinase inhibitor (and optionally yet a further combination partner (e.g. an other drug as explained below, also referred to as “co-agent”) may be administered independently at the same time or separately within time intervals, especially where these time intervals allow that the combination partners show a cooperative (=joint), e.g. synergistic effect. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration and/or at the same time. The term “combination product” as used herein thus means a pharmaceutical product that results from the mixing or combining of more than one active ingredient and includes both fixed and non-fixed combinations of the active ingredients (which may also be combined).

The term “fixed combination” means that the active ingredients, e.g. an EGFR tyrosine kinase inhibitor and MET tyrosine kinase inhibitor, are both administered to a patient simultaneously in the form of a single entity or dosage. In other terms: the active ingredients are present in one dosage form, e.g. in one tablet or in one capsule.

The term “non-fixed combination” means that the active ingredients are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients. The term “non-fixed combination” thus defines especially a “kit of parts” in the sense that the combination partners (i) EGFR tyrosine kinase inhibitor and (ii) MET tyrosine kinase inhibitor (and if present further one or more co-agents) as defined herein can be dosed independently of each other or by use of different fixed combinations with distinguished amounts of the combination partners, i.e. simultaneously or at different time points, where the combination partners may also be used as entirely separate pharmaceutical dosage forms or pharmaceutical formulations that are also sold independently of each other and just instructions of the possibility of their combined use is or are provided in the package equipment, e.g. leaflet or the like, or In other information e.g. provided to physicians and medical staff. The independent formulations or the parts of the kit of parts can then, e.g. be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (i) and (ii), thus being jointly active. The ratio of the total amounts of the combination partner (i) to the combination partner (ii) to be administered in the combined preparation can be varied, e.g. In order to cope with the needs of a patient sub-population to be treated or the needs of the single patient which different needs can be due to age, sex, body weight, etc. of the patients.

The disclosure also relates to (i) a MET inhibitor which is INC280 or a pharmaceutically acceptable salt thereof and (ii) an EGFR inhibitor which is a monoclonal antibody, for combined use in a method of treating an EGFR tyrosine kinase activity and/or MET tyrosine kinase activity mediated disease, especially a cancer.

The combination partners (i) and (ii) In any embodiment are preferably formulated or used to be jointly (prophylactically or especially therapeutically) active. This means in particular that there is at least one beneficial effect, e.g. a mutual enhancing of the effect of the combination partners (i) and (ii), in particular a synergism, e.g. a more than additive effect, additional advantageous effects (e.g. a further therapeutic effect not found for any of the single compounds), less side effects, a combined therapeutic effect in a non-effective dosage of one or both of the combination partners (i) and (ii), and very preferably a clear synergism of the combination partners (i) and (ii). For example, the term “jointly (therapeutically) active” may mean that the compounds may be given separately or sequentially (In a chronically staggered manner, especially a sequence-specific manner) In such time intervals that they preferably, in the warm-blooded animal, especially human, to be treated, and still show a (preferably synergistic) interaction (joint therapeutic effect). A joint therapeutic effect can, inter alia, be determined by following the blood levels, showing that both compounds are present in the blood of the human to be treated at least during certain time intervals, but this is not to exclude the case where the compounds are jointly active although they are not present in blood simultaneously.

The present disclosure thus pertains to a combination product for simultaneous, separate or sequential use, such as a combined preparation or a pharmaceutical fixed combination, or a combination of such preparation and combination.

In the combination therapies of the disclosure, the compounds useful according to the disclosure may be manufactured and/or formulated by the same or different manufacturers. Moreover, the combination partners may be brought together into a combination therapy: (i) prior to release of the combination product to physicians (e.g. in the case of a kit comprising the compound of the disclosure and the other therapeutic agent); (ii) by the physician themselves (or under the guidance of a physician) shortly before administration; (iii) In the patient themselves, e.g. during sequential administration of the compound of the disclosure and the other therapeutic agent.

In certain embodiments, any of the above methods involve further administering one or more other (e.g. third) co-agents, especially a chemotherapeutic agent.

Thus, the disclosure relates in a further embodiment to a combination product, particularly a pharmaceutical composition, comprising a therapeutically effective amount of (i) an EGFR tyrosine kinase inhibitor which is a monoclonal antibody and (ii) a MET tyrosine kinase inhibitor which is INC280 or a pharmaceutically acceptable salt thereof, and at least one third therapeutically active agent (co-agent), e.g. another compound (i) and/or (ii) or a different co-agent. The additional co-agent is preferably selected from the group consisting of an anti-cancer agent; an anti-inflammatory agent.

Also in this case, the combination partners forming a corresponding product according to the disclosure may be mixed to form a fixed pharmaceutical composition or they may be administered separately or pairwise (i.e. before, simultaneously with or after the other drug substance(s)).

A combination product according to the disclosure can besides or In addition be administered especially for cancer therapy in combination with chemotherapy, radiotherapy, immunotherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Possible anti-cancer agents (e.g. for chemotherapy) as co-agents include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity; anti-angiogenic compounds; compounds which target, decrease or inhibit the activity of a protein or lipid phosphatase; gonadorelin agonists; anti-androgens; methionine aminopeptidase inhibitors; bisphosphonates; biological response modifiers; antiproliferative antibodies; heparanase inhibitors; Inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors; kinesin spindle protein inhibitors; MEK inhibitors; leucovorin; EDG binders; antileukemia compounds; ribonucleotide reductase inhibitors; S-adenosylmethionine decarboxylase inhibitors; angiostatic steroids; corticosteroids; other chemotherapeutic compounds (as defined below); photosensitizing compounds.

Further, alternatively or in addition combination products according to the disclosure may be used in combination with other tumor treatment approaches, including surgery, ionizing radiation, photodynamic therapy, implants, e.g. with corticosteroids, hormones, or they may be used as radiosensitizers.

The term “aromatase Inhibitor” as used herein relates to a compound which inhibits the estrogen production, i.e. the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole.

The term “antiestrogen” as used herein relates to a compound which antagonizes the effect of estrogens at the estrogen receptor level. The term includes, but is not limited to tamoxifen, fulvestrant, raloxifene and raloxifene hydrochloride.

The term “anti-androgen” as used herein relates to any substance which is capable of inhibiting the biological effects of androgenic hormones and includes, but is not limited to, bicalutamide (CASODEX), which can be formulated, e.g. as disclosed in U.S. Pat. No. 4,636,505.

The term “gonadorelin agonist” as used herein includes, but is not limited to abarelix, goserelin and goserelin acetate. The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148 (compound A1 in WO99/17804).

The term “topoisomerase II inhibitor” as used herein includes, but is not limited to the anthracyclines such as doxorubicin (including liposomal formulation, e.g. CAELYX), daunorubicin, epirubicin, idarubicin and nemorubicin, the anthraquinones mitoxantrone and losaxantrone, and the podophillotoxines etoposide and teniposide.

The term “microtubule active compound” relates to microtubule stabilizing, microtubule destabilizing compounds and microtublin polymerization inhibitors including, but not limited to taxanes, e.g. paclitaxel and docetaxel, vinca alkaloids, e.g., vinblastine, especially vinblastine sulfate, vincristine especially vincristine sulfate, and vinorelbine, discodermolides, cochicine and epothilones and derivatives thereof, e.g. epothilone B or D or derivatives thereof.

The term “alkylating compound” as used herein includes, but is not limited to, cyclophosphamide, ifosfamide, melphalan or nitrosourea (BCNU or Gliadel).

The term “histone deacetylase inhibitors” or “HDAC Inhibitors” relates to compounds which inhibit the histone deacetylase and which possess antiproliferative activity. This includes compounds disclosed in WO 02/22577, especially N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(2-methyl-1H-indol-3-yl)-ethyl]-amino]methyl]phenyl]-2E-2-propenamide and pharmaceutically acceptable salts thereof. It further especially includes Suberoylanilide hydroxamic acid (SAHA).

Compounds which target, decrease or inhibit activity of histone deacetylase (HDAC) Inhibitors such as sodium butyrate and suberoylanilide hydroxamic acid (SAHA) Inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065, in particular, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl)[2-(1H-indol-3-yl)ethyl]-amino]methyl]phenyl]-2E-2-propenamide, or a pharmaceutically acceptable salt thereof, especially the lactate salt.

The term “antineoplastic antimetabolite” includes, but is not limited to, 5-Fluorouracil or 5-FU, capecitabine, gemcitabine, DNA demethylating compounds, such as 5-azacytidine and decitabine, methotrexate and edatrexate, and folic acid antagonists such as pemetrexed.

The term “platin compound” as used herein includes, but is not limited to, carboplatin, ds-platin, cisplatinum and oxaliplatin.

The term “compounds targeting/decreasing a protein or lipid kinase activity”; or a “protein or lipid phosphatase activity”; or “further anti-angiogenic compounds” as used herein includes, but is not limited to, c-Met tyrosine kinase and/or serine and/or threonine kinase inhibitors or lipid kinase inhibitors, e.g.,

a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, e.g. a N-phenyl-2-pyrimidine-amine derivative, e.g. imatinib, SU101, SU6668 and GFB-111;
b) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, such as those compounds disclosed in WO 02/092599, or antibodies that target the extracellular domain of IGF-I receptor or its growth factors;
c) compounds targeting, decreasing or inhibiting the activity of the Trk receptor tyrosine kinase family, or ephrin kinase family inhibitors;
d) compounds targeting, decreasing or inhibiting the activity of the Axl receptor tyrosine kinase family;
e) compounds targeting, decreasing or inhibiting the activity of the Ret receptor tyrosine kinase;
f) compounds targeting, decreasing or inhibiting the activity of the Kit/SCFR receptor tyrosine kinase, e.g. imatinib;
g) compounds targeting, decreasing or inhibiting the activity of the C-kit receptor tyrosine kinases—(part of the PDGFR family), such as compounds which target, decrease or inhibit the activity of the c-Kit receptor tyrosine kinase family, especially compounds which inhibit the c-Kit receptor, e.g. Imatinib;
h) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, e.g. a N-phenyl-2-pyrimidine-amine derivative, e.g. imatinib or nilotinib (AMN107); PD180970; AG957; NSC 680410; PD173955 from ParkeDavis; or dasatinib (BMS-354825)
i) compounds targeting, decreasing or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK) and are especially those staurosporine derivatives disclosed in U.S. Pat. No. 5,093,330, e.g. midostaurin; examples of further compounds include e.g. UCN-01, safingol, BAY 43-9006, Bryostatin 1, Perifosine; limofosine; RO 318220 and RO 320432; GO 6976; isis 3521; LY333531/LY379196; isochinoline compounds such as those disclosed in WO 00/09495; FTIs; PD184352 or QAN697 (a P13K inhibitor) or AT7519 (CDK inhibitor);
j) compounds targeting, decreasing or inhibiting the activity of protein-tyrosine kinase inhibitors, such as compounds which target, decrease or inhibit the activity of protein-tyrosine kinase inhibitors include imatinib mesylate (GLEEVEC) or tyriphostin. A tyrphostin is preferably a low molecular weight (Mr<1500) compound, or a pharmaceutically acceptable salt thereof, especially a compound selected from the benzylidenemalonitrile class or the S-arylbenzenemalonirile or bisubstrate quinoline class of compounds, more especially any compound selected from the group consisting of Tyrphostin A23/RG-50810; AG 99; Tyrphostin AG 213; Tyrphostin AG 1748; Tyrphostin AG 490; Tyrphostin B44; Tyrphostin B44 (+) enantiomer, Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin);
k) compounds targeting, decreasing or inhibiting the activity of the epidermal growth factor family of receptor tyrosine kinases (EGFR, ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, e.g. EGF receptor, ErbB2, ErbB3 and ErbB4 or bind to EGF or EGF related ligands, and are in particular those compounds, proteins or monoclonal antibodies generically and specifically disclosed in WO 97/02266, e.g. the compound of ex. 39, or in EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/30347 (e.g. compound known as CP 358774), WO 96/33980 (e.g. compound ZD 1839) and WO 95/03283 (e.g. compound ZM105180); e.g. trastuzumab (Herceptin™), cetuximab (Erbitux™), Iressa, Tarceva, OSI-774, CI-1033, EKB-569, GW-2016, E1.1, E2.4, E2.5, E6.2, E6.4, E2.11, E6.3 or E7.6.3, and 7H-pyrrolo-[2,3-d]pyrimidine derivatives which are disclosed in WO 03/013541; and
l) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF;
m) compounds targeting, decreasing or inhibiting the activity of the Ron receptor tyrosine kinase.

Further anti-angiogenic compounds include compounds having another mechanism for their activity, e.g. unrelated to protein or lipid kinase inhibition e.g. thalidomide (THALOMID) and TNP-470.

The term “Compounds which target, decrease or inhibit the activity of a protein or lipid phosphatase” includes, but is not limited to inhibitors of phosphatase 1, phosphatase 2A, or CDC25, e.g. okadaic acid or a derivative thereof. The term “Compounds which induce cell differentiation processes” includes, but is not limited to e.g. retinoic acid, α- γ- or δ-tocopherol or α- γ- or δ-tocotrienol.

The term “cyclooxygenase inhibitor” as used herein includes, but is not limited to, e.g. Cox-2 Inhibitors, 5-alkyl substituted 2-arylaminophenylacetic acid and derivatives, such as celecoxib (CELEBREX), rofecoxib (VIOXX), etoricoxib, valdecoxib or a 5-alkyl-2-arylaminophenylacetic acid, e.g. 5-methyl-2-(2′-chloro-6′-fluoroanilino)phenyl acetic acid, lumiracoxib The term “bisphosphonates” as used herein includes, but is not limited to, etridonic, clodronic, tiludronic, pamidronic, alendronic, ibandronic, risedronic and zoledronic acid.

The term “mTOR inhibitors” relates to compounds which inhibit the mammalian target of rapamycin (mTOR) and which possess antiproliferative activity such as sirolimus (Rapamune®), everolimus (Certican™), CC-779 and ABT578.

The term “heparanase inhibitor” as used herein refers to compounds which target, decrease or inhibit heparin sulfate degradation. The term includes, but is not limited to, PI-88.

The term “biological response modifier” as used herein refers to a lymphokine or interferons, e.g. interferon γ.

The term “Inhibitor of Ras oncogenic isoforms”, e.g. H-Ras, K-Ras, or N-Ras, as used herein refers to compounds which target, decrease or inhibit the oncogenic activity of Ras e.g. a “farnesyl transferase inhibitor” e.g. L-744832, DK8G557 or R115777 (Zamestra).

The term “telomerase inhibitor” as used herein refers to compounds which target, decrease or inhibit the activity of telomerase. Compounds which target, decrease or inhibit the activity of telomerase are especially compounds which inhibit the telomerase receptor, e.g. telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase are e.g. bengamide or a derivative thereof.

The term “proteasome inhibitor” as used herein refers to compounds which target, decrease or inhibit the activity of the proteasome. Compounds which target, decrease or inhibit the activity of the proteasome include e.g. Bortezomid (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors e.g. compounds targeting, decreasing or inhibiting the activity of FMS-like tyrosine kinase receptors (Flt-3R); interferon, 1-b-D-arabinofuransylcytosine (ara-c) and bisulfan; and ALK inhibitors e.g. compounds which target, decrease or inhibit anaplastic lymphoma kinase.

The term “Compounds which target, decrease or inhibit the activity of FMS-like tyrosine kinase receptors (Flt-3R)” are especially compounds, proteins or antibodies which inhibit members of the Flt-3R receptor kinase family, e.g. PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90 e.g., 17-allylamino, 17-demethoxygeldanamycin (17AAG, 17-DMAG), a geldanamycin derivative; other geldanamycin related compounds; radicicol and HDAC inhibitors; IPI-504, CNF1010, CNF2024, CNF1010 from Conforma Therapeutics; temozolomide, AUY922 from Novartis.

The term “antiproliferative antibodies” as used herein includes, but is not limited to erbitux, bevacidzumab, rituximab, PRO64553 (anti-CD40) and 2C4 Antibody. By antibodies is meant e.g. intact monoclonal antibodies, polyclonal antibodies, multispecific antibodies formed from at least 2 Intact antibodies, and antibodies fragments so long as they exhibit the desired biological activity.

The term “antileukemic compounds” includes, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. For the treatment of acute myeloid leukemia (AML), compounds of formula (I) can be used in combination with standard leukemia therapies, especially in combination with therapies used for the treatment of AML. In particular, compounds of formula (I) can be administered in combination with, e.g., farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

“Somatostatin receptor antagonists” as used herein refers to compounds which target, treat or inhibit the somatostatin receptor such as octreotide, and SOM230.

“Tumor cell damaging approaches” refer to approaches such as Ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

The term “EDG binders” as used herein refers a class of immunosuppressants that modulates lymphocyte recirculation, such as FTY720.

The term “kinesin spindle protein inhibitors” is known in the field and includes SB715992 or SB743921 from GlexoSmithKline, pentamidine/chlorpromazine from CombinatoRx.

The term “MEK inhibitors” is known in the field and includes ARRY142886 from Array PioPharma, AZD6244 from AstraZeneca, PD181461 from Pfizer, leucovorin.

The term “ribonucleotide reductase inhibitors” Includes, but is not limited to to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives, such as PL-1, PL-2, PL-3, PL-4, PL-5, PL-6, PL-7 or PL-8 mentioned in Nandy et al., Acta Oncologica, Vol. 33, No. 8, pp. 953-961 (1994).

The term “S-adenosylmethionine decarboxylase inhibitors” as used herein includes, but is not limited to the compounds disclosed in U.S. Pat. No. 5,461,076.

Also included are in particular those compounds, proteins or monoclonal antibodies of VEGF/VEGFR disclosed in WO 98/35958, e.g. 1-(4-chloroanilino)-4-(4-pyridylmethyl)phthalazine or a pharmaceutically acceptable salt thereof, e.g. the succinate, or In WO 00/09495, WO 00/27820, WO 00/59509. WO 98/11223, WO 00/27819 and EP 0 769 947; those as described by Prewett et al, Cancer Res, Vol. 59, pp. 5209-5218 (1999); Yuan et al., Proc Natl Acad Sci USA, Vol. 93, pp. 14765-14770 (1996); Zhu et al., Cancer Res, Vol. 58, pp. 3209-3214 (1998); and Mordenti at al., Toxicol Pathol, Vol. 27, No. 1, pp. 14-21 (1999); in WO 00/37502 and WO 94/10202; ANGIOSTATIN, described by O'Reilly at al., Cell, Vol. 79, pp. 315-328 (1994); ENDOSTATIN, described by O'Reilly at al., Cell, Vol. 88, pp. 277-285 (1997); anthranilic acid amides; ZD4190; ZD6474; SU5416; SU6668; bevacizumab; or anti-VEGF antibodies or anti-VEGF receptor antibodies, e.g. rhuMAb and RHUFab, VEGF aptamer e.g. Macugon; FLT-4 inhibitors, FLT-3 Inhibitors, VEGFR-2 IgG1 antibody, Angiozyme (RPI 4610) and Bevacizumab.

“Photodynamic therapy” as used herein refers to therapy which uses certain chemicals known as photosensitizing compounds to treat or prevent cancers. Examples of photodynamic therapy includes treatment with compounds, such as e.g. VISUDYNE and porfimer sodium.

“Angiostatic steroids” as used herein refers to compounds which block or inhibit angiogenesis, such as, e.g., anecortave, triamcinolone. hydrocortisone, 11-α-epihydrocotisol, cortexolone, 17α-hydroxyprogesterone, corticosterone, desoxycorticosterone, testosterone, estrone and dexamethasone.

“Corticosteroids” as used herein includes, but is not limited to compounds, such as e.g. fluocinolone, dexamethasone; in particular in the form of implants.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

A combination product according to the disclosure may also be used in combination with or comprise one or more further drug substances selected from the group of anti-inflammatory drug substances; antihistamine drug substances; bronchodilatatory drug substances, NSAID; antagonists of chemokine receptors.

Suitable anti-inflammatory drugs include steroids, in particular glucocorticosteroids such as budesonide, beclamethasone dipropionate, fluticasone propionate, ciclesonide or mometasone furoate, or steroids described in WO 02/88167, WO 02/12266, WO 02/100879, WO 02/00679 (especially those of Examples 3, 11, 14, 17, 19, 26, 34, 37, 39, 51, 60, 67, 72, 73, 90, 99 and 101), WO 03/035668, WO 03/048181, WO 03/062259, WO 03/064445, WO 03/072592, non-steroidal glucocorticold receptor agonists such as those described in WO 00/00531, WO 02/10143, WO 03/082280, WO 03/082787, WO 03/104195, and WO 04/005229.

LTB4 antagonists such LY293111, CGS025019C, CP-195543, SC-53228, BIIL 284, ONO 4057, SB 209247 and those described in U.S. Pat. No. 5,451,700; LTD4 antagonists such as montelukast and zafirkukast; PDE4 inhibitors such as cilomilast, Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medics), CDC-801 (Celgene), SelCID™ CC-10004 (Celgene), VM554/UM565 (Vernalis), T-440 (Tanabe), KW-4490 (Kyowa Hakko Kogyo), and those disclosed in WO 92/19594, WO 93/19749, WO 93/19750, WO 93/19751. WO 98/18796, WO 99/16766, WO 01/13953, WO 03/104204, WO 03/104205, WO 03/39544, WO 04/000814, WO 04/000839, WO 04/005258, WO 04/018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607 and WO 04/037805; A2a agonists such as those disclosed in EP 409595A2, EP 1052264, EP 1241176, WO 94/17090, WO 96/02543, WO 96/02553, WO 98/28319, WO 99/24449, WO 99/24450, WO 99/24451, WO 99/38877, WO 99/41287, WO 99/67263, WO 99/67264, WO 99/67265, WO 99/67268, WO 00/23457, WO 00/77018, WO 00/78774, WO 01/23399, WO 01/27130, WO 01/27131, WO 01/60835, WO 01/94368, WO 02/00676, WO 02/22630, WO 02/96462, WO 03/086408, WO 04/039762, WO 04/039766, WO 04/045618 and WO 04/046083; A2b antagonists such as those described in WO 02/42298; and beta-2 adrenoceptor agonists such as albuterol (salbutamol), metaproterenol, terbutaline, salmeterol fenoterol, procaterol, and especially, formoterol and pharmaceutically acceptable salts thereof, and compounds (In free or salt or solvate form) of formula I of WO 0075114, which document is Incorporated herein by reference, preferably compounds of the Examples thereof, especially a compound of formula

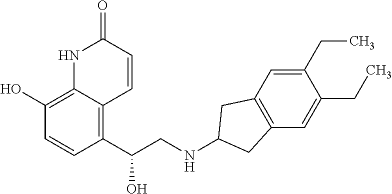 [SMILES: CCc1cc2c(cc1CC)CC(NC[C@H](O)c1ccc(O)c3[nH]c(=O)ccc13)C2]

and pharmaceutically acceptable salts thereof, as well as compounds (in free or salt or solvate form) of formula I of WO 04/16601, and also compounds of WO 04/033412.

Suitable bronchodilatory drugs include anticholinergic or antimuscarinic compounds, in particular ipratropium bromide, oxitropium bromide, tiotropium salts and CHF 4226 (Chiesi), and glycopyrrolate, but also those described in WO 01/04118, WO 02/51841, WO 02/53564, WO 03/00840, WO 03/87094, WO 04/05285, WO 02/00652, WO 03/53966, EP 424021, U.S. Pat. Nos. 5,171,744, 3,714,357, WO 03/33495 and WO 04/018422.

Suitable chemokine receptors include, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, Takeda antagonists such as N-[[4-[[[6,7-dihydro-2-(4-methylphenyl)-5H-benzocyclohepten-8-yl]carbonyl]amino]phenyl]-methyl]tetrahydro-N, N-dimethyl-2H-pyran-4-amin-ium chloride (TAK-770), and CCR-5 antagonists described in U.S. Pat. No. 6,166,037 (particularly claims 18 and 19), WO 00/66558 (particularly claim 8), WO 00/66559 (particularly claim 9), WO 04/018425 and WO 04/026873.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine as well as those disclosed in WO 03/099807, WO 04/026841 and JP 2004107299.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

The term “pharmaceutically effective” preferably relates to an amount that is therapeutically or in a broader sense also prophylactically effective against the progression of a disease or disorder as disclosed herein.

The term “a commercial package” as used herein defines especially a “kit of parts” in the sense that the components (a) MET tyrosine kinase inhibitor and (b) EGFR tyrosine kinase inhibitor as defined above and below, and optionally further co-agents, can be dosed independently or by use of different fixed combinations with distinguished amounts of the components (a) and (b), i.e., simultaneously or at different time points. Moreover, these terms comprise a commercial package comprising (especially combining) as active ingredients components (a) and (b), together with instructions for simultaneous, sequential (chronically staggered, in time-specific sequence, preferentially) or (less preferably) separate use thereof in the delay of progression or treatment of a proliferative disease. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (a) and (b) (as can be determined according to standard methods. The ratio of the total amounts of the combination partner (a) to the combination partner (b) to be administered in the combined preparation can be varied, e.g., in order to cope with the needs of a patient sub-population to be treated or the needs of the single patient which different needs can be due to the particular disease, age, sex, body weight, etc. of the patients. Preferably, there is at least one beneficial effect, e.g., a mutual enhancing of the effect of the combination partners (a) and (b), in particular a more than additive effect, which hence could be achieved with lower doses of each of the combined drugs, respectively, than tolerable in the case of treatment with the individual drugs only without combination, producing additional advantageous effects, e.g., less side effects or a combined therapeutic effect in a non-effective dosage of one or both of the combination partners (components) (a) and (b), and very preferably a strong synergism of the combination partners (a) and (b).

Both in the case of the use of the combination of components (a) and (b) and of the commercial package, any combination of simultaneous, sequential and separate use is also possible, meaning that the components (a) and (b) may be administered at one time point simultaneously, followed by administration of only one component with lower host toxicity either chronically, e.g., more than 3-4 weeks of daily dosing, at a later time point and subsequently the other component or the combination of both components at a still later time point (in subsequent drug combination treatment courses for an optimal effect) or the like.

The combination products according to the present disclosure are appropriate for the treatment of various diseases that are mediated by, especially depend on, the activity of EGFR and/or MET tyrosine kinase, respectively. They can thus be used in the treatment of any of the diseases that can be treated by EGFR tyrosine kinase inhibitors and MET tyrosine kinase inhibitors.

EGFR inhibitors are e.g. useful in the treatment of one or more of the diseases which respond to an inhibition of EGFR activity, especially a neoplastic or tumor disease, especially solid tumor, more especially those cancers in which EGFR kinases are implicated including breast cancer, gastric cancer, lung cancer, cancer of the prostate, bladder cancer and endometrial cancer. Further cancers include cancer of the kidney, liver, adrenal glands, stomach, ovaries, colon, rectum, pancreas, vagina or thyroid, sarcoma, glioblastomas and numerous tumours of the neck and head, as well as leukemias and multiple myeloma. Especially preferred are cancers of breast or ovary; lung cancer, e.g. NSCLC or SCLC; head and neck, renal, colorectal, pancreas, bladder, gastric or prostate cancer; or glioma; in particular, glioma or colon, rectum or colorectal cancer or more particularly lung cancer are to be mentioned. Also diseases dependent on ligands of EGFR, such as EGF; TGF-α; HB-EGF; amphiregulin; epiregulin; betacellulin, are included.

MET inhibitors are e.g. useful in the treatment of MET related diseases, especially cancers that display evidence for simultaneous activation of MET and FGFR, including gene amplification, activating mutations, expression of cognate RTK ligands, phosphorylation of RTKs at residues indicative of activation, e.g. where the cancer is selected from the group consisting of brain cancer, stomach cancer, genital cancer, urinary cancer, prostate cancer, (urinary) bladder cancer (superficial and muscle invasive), breast cancer, cervical cancer, colon cancer, colorectal cancer, glioma (including glioblastoma, anaplastic astrocytoma, oligoastrocytoma, oligodendroglioma), esophageal cancer, gastric cancer, gastrointestinal cancer, liver cancer, hepatocellular carcinoma (HCC) including childhood HCC, head and neck cancer (including head and neck squamous-cell carcinoma, nasopharyngeal carcinoma), Hurthle cell carcinoma, epithelial cancer, skin cancer, melanoma (including malignant melanoma), mesothelioma, lymphoma, myeloma (including multiple myeloma), leukemias, lung cancer (including non-small cell lung cancer (Including all histological subtypes: adenocarcinoma, squamous cell carcinoma, bronchoalveolar carcinoma, large-cell carcinoma, and adenosquamous mixed type), small-cell lung cancer), ovarian cancer, pancreatic cancer, prostate cancer, kidney cancer (including but not limited to papillary renal cell carcinoma), intestine cancer, renal cell cancer (Including hereditary and sporadic papillary renal cell cancer, Type I and Type II, and clear cell renal cell cancer); sarcomas, in particular osteosarcomas, clear cell sarcomas, and soft tissue sarcomas (including alveolar and (e.g. embryonal) rhabdomyosarcomas, alveolar soft part sarcomas); thyroid carcinoma (papillary and other subtypes).

MET inhibitors are e.g. also useful in the treatment of cancer wherein the cancer is stomach, colon, liver, genital, urinary, melanoma, or prostate. In a particular embodiment, the cancer is liver or esophageal.

MET inhibitors are e.g. also useful in the treatment of colon cancer, including metastases, e.g. In the liver, and of non-small-cell lung carcinoma.

MET inhibitors are e.g. also may be used in the treatment of hereditary papillary renal carcinoma (Schmidt, L. et al. Nat. Genet. 16, 68-73, 1997) and other proliferative diseases in which c-MET is overexpressed or constitutively activated by mutations (Jeffers and Vande Woude. Oncogene 18, 5120-5125, 1999; and reference cited therein) or chromosomal rearrange-ments (e.g. TPR-MET; Cooper at al. Nature 311, 29-33, 1984; Park. et al. Cell 45, 895-904, 1988).

MET inhibitors are e.g. further useful in the treatment of additional cancers and conditions as provided herein or known in the art.

MET inhibitors are e.g. also suitable for the treatment of one or more inflammatory conditions.

In a further embodiment, the inflammatory condition is due to an infection. In one embodiment, the method of treatment would be to block pathogen infection. In a particular embodiment, the infection is a bacterial infection, e.g., a Listeria Infection. See, e.g., Shen at al. Cell 103: 501-10, (2000) whereby a bacterial surface protein activates c-Met kinase through binding to the extracellular domain of the receptor, thereby mimicking the effect of the cognate ligand HGF/SF.

The combination product of the present disclosure is especially appropriate for treatment of any of the cancers mentioned above amenable to EGFR or Met Inhibitor treatment, especially a cancer selected from adenocarcinoma (especially of the breast or more especially of the lung), rhabdomyosarcoma, osteosarcoma, urinary bladder carcinoma, colorectal cancer and glioma.

The term “a therapeutically effective amount” of a compound of the present disclosure refers to an amount of the compound of the present disclosure that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present disclosure that, when administered to a subject, is effective to (1) at least partially alleviating, inhibiting, preventing and/or ameliorating a condition, or a disorder or a disease (i) mediated by cMet (MET) and/or mediated by EGFR activity, or (ii) characterized by activity (normal or abnormal) of cMet and/or of EGFR; or (2) reducing or inhibiting the activity of cMet and/or of EGFR; or (3) reducing or inhibiting the expression of cMet and/or EGFR. In another non-limiting embodiment, the term “a therapeutically effective amount” refers to the amount of the compound of the present disclosure that, when administered to a cell, or a tissue, or a non-cellular biological material, or a medium, is effective to at least partially reducing or inhibiting the activity of cMet and/or EGFR; or at least partially reducing or inhibiting the expression of MET and/or EGFR.

As used herein, the term “subject” refers to an animal. Typically the animal is a mammal. A subject also refers to for example, primates (e.g., humans), cows, sheep, goats, horses, dogs, cats, rabbits, rats, mice, fish, birds and the like. In certain embodiments, the subject is a primate. In yet other embodiments, the subject is a human.

“And/or” means that each one or both or all of the components or features of a list are possible variants, especially two or more thereof in an alternative or cumulative way.

As used herein, the term “inhibit”, “Inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

The term “treatment” comprises, for example, the prophylactic or especially therapeutic administration of the combination partners to a warm-blooded animal, preferably to a human being, in need of such treatment with the aim to cure the disease or to have an effect on disease regression or on the delay of progression of a disease.

As used herein, a subject is “in need of” a treatment if such subject would benefit biologically, medically or in quality of life from such treatment.

As used herein, the term “a,” “an,” “the” and similar terms used in the context of the present disclosure (especially in the context of the claims) are to be construed to cover both the singular and plural unless otherwise indicated herein or clearly contradicted by the context.

The combinations according to the disclosure can be prepared in a manner known per se and are those suitable for enteral, such as oral or rectal, and parenteral administration to mammals (warm-blooded animals), including man, comprising a therapeutically effective amount of at least one pharmacologically active combination partner alone or in combination with one or more pharmaceutically acceptable carriers, especially suitable for enteral or parenteral application. In one embodiment of the disclosure, one or more of the active ingredients are administered orally.

As used herein, the term “carrier” or “pharmaceutically acceptable carrier” includes any and al solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The pharmaceutical combination product according to the disclosure (as fixed combination, or as kit, e.g. as combination of a fixed combination and individual formulations for one or both combination partners or as kit of individual formulations of the combination partners) comprises the combination partners (at least one MET tyrosine kinase inhibitor, at least one EGFR tyrosine kinase inhibitor, and optionally one or more further co-agents) of the present disclosure and one or more pharmaceutically acceptable carrier materials (carriers, excipients).

The combination products or the combination partners constituting it can be formulated for particular routes of administration such as oral administration, parenteral administration, and rectal administration, etc. In addition, the combination products of the present disclosure can be made up in a solid form (Including without imitation capsules, tablets, pills, granules, powders or suppositories), or in a liquid form (including without limitation solutions, suspensions or emulsions). The combination products and/or their combination partners can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

In one embodiment, the pharmaceutical compositions are tablets or gelatin capsules comprising the active ingredient together with one or more commonly known carriers, e.g. one or more carriers selected from the group consisting of
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and
e) absorbents, colorants, flavors and sweeteners.
   Tablets may be either film coated or enteric coated according to methods known in the art.

Suitable compositions for oral administration especially include an effective amount of one or more or In case of fixed combination formulations each of the combination partners (active ingredients) in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient(s) in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient Is mixed with water or an oil medium, for example, peanut oil, liquid paraffin or olive oil.

Certain injectable compositions (especially useful e.g. where antibodies are used as EGFR inhibitors) are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of one or more active ingredients with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

The disclosure relates also to a kit of parts or a fixed pharmaceutical composition comprising an effective amount, especially an amount effective in the treatment of one of the abovementioned diseases of at least one MET tyrosine kinase inhibitor, at least one EGFR tyrosine kinase inhibitor, or a pharmaceutically acceptable salt thereof, respectively, and optionally of at least one further co-agent, or a pharmaceutically acceptable salt thereof, together with one or more pharmaceutically acceptable carriers that are suitable for topical, enteral, for example oral or rectal, or parenteral administration and that may be inorganic or organic, solid or liquid.

In all formulations, the active ingredient(s) forming part of a combination product according to the present disclosure can be present each in a relative amount of 0.5 to 95% of weight of the corresponding formulation (regarding the formulation as such, that is without packaging and leaflet), e.g. from 1 to 90, 5 to 95, 10 to 98 or 10 to 60 or 40 to 80% by weight, respectively.

The dosage of the active ingredient to be applied to a warm-blooded animal depends upon a variety of factors including type, species, age, weight, sex and medical condition of the patient; the severity of the condition to be treated; the route of administration; the renal and hepatic function of the patient; and the particular compound employed. A physician, clinician or veterinarian of ordinary skill can readily determine and prescribe the effective amount of the drug required to prevent, counter or arrest the progress of the condition. Optimal precision in achieving concentration of drug within the range that yields efficacy without toxicity requires a regimen based on the kinetics of the drug's availability to target sites. This involves a consideration of the distribution, equilibrium, and elimination of a drug. The dose of each of the combination partners or a pharmaceutically acceptable salt thereof to be administered to warm-blooded animals, for example humans of approximately 70 kg body weight, is preferably from approximately 3 mg to approximately 5 g, more preferably from approximately 10 mg to approximately 1.5 g per person per day, e.g. divided preferably into 1 to 3 single doses, e.g. for use once or twice daily, which may, for example, be of the same size. Usually, children receive half of the adult dose.

The pharmaceutical combination product of the present disclosure can e.g. be in unit dosage of about 1-1000 mg of active ingredient(s) for a subject of about 50-70 kg, or about 1-500 mg or about 1-250 mg or about 1-150 mg or about 0.5-100 mg, or about 1-50 mg of for any one or in particular the sum of active ingredients; or (especially for the EGFR inhibitor) 50 to 900, 60 to 850, 75 to 800 or 100 to 600 mg, respectively, for any one or in particular the sum of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or (in animal use) veterinarian of ordinary skill can readily determine the effective amount of each of the active ingredients necessary to prevent, treat or inhibit the progress of the disorder or disease.

Specific embodiments of the disclosure are also given in the claims which are incorporated here by reference, as well as in the Examples.

### EXAMPLES

The following Examples illustrate the disclosure and provide specific embodiments, however without limiting the scope of the disclosure.

| Abbreviation | Description |
|---|---|
| EGFR | Epidermal Growth Factor Receptor |
| HGF | Hepatocyte Growth Factor |
| FBS | Fetal Bovine Serum |
| PBS | Phosphate Buffered Saline |
| IC50 | 50% Inhibitory Concentration |
| CTG | Cell Titer Glo |
| RTCA | Real Time Cell Proliferation Assay |
| CRC | Colorectal Carcinoma |
| HNSCC | Head and Neck Squamous Cell Carcinomas |
| Combo | Combination |
| Cetu | Cetuximab |
| CCLE | Cancer Cell Line Encyclopedia |

### Example 1: Combination of cMET Inhibitor INC280 and EGFR Inhibitor Cetuximab in Colon and Head & Neck Cancer Cell Lines

In this study, hepatocyte growth factor (HGF) rescue of growth inhibition by Cetuximab in head and neck squamous cell carcinoma (HNSCC) and colorectal (CRC) cancer cell lines and the ability of MET inhibitor INC280 to block the HGF effect were demonstrated using 3 day CTG assay. In addition, the anti-proliferation activity of INC280 and Cetuximab combination in YD38, CAL-33 and CCK-81 cells was assessed, both in the absence and presence of exogenous HGF. The combination studies were conducted with a “dose matrix”, where the combination was tested in all possible permutations of serially-diluted Cetuximab and INC280: Cetuximab was subjected to a 8 dose 3× serial dilution with highest dose at 0.3 μM and lowest dose at about 0.4 nM and INC280 was subjected to a 8 dose 3× serial dilution with the highest dose at 1.5 μM and the lowest dose at about 2 nM. Cetuximab single agent showed a potent and concentration-dependent activity of inhibiting proliferation of YD-38, CAL-33 and CCK-81 cells, and addition of HGF to those cells abolished the activity of Cetuximab at nearly all concentrations. As low as 18 nM INC280 re-sensitized cells to Cetuximab in the presence of HGF and the INC280/Cetuximab combination was highly synergistic (synergy scores ranging from 4.3 to 14.0, using a dose additive synergistic model). Importantly, the combination synergy was only observed in the presence of HGF but not in the absence of HGF. INC280 as a single agent had little or no anti-proliferation effect to those three cell lines regardless of HGF addition. In conclusion, combining INC280 with Cetuximab may potentially both overcome the intrinsic resistance and prevent the acquired resistance to Cetuximab mediated by various types of MET activation such as HGF overexpression or MET amplification in HNSCC and CRC tumors.

MET amplification or hepatocyte growth factor (HGF) overexpression has been implicated in the acquired resistance of lung cancer to EGFR inhibitors such as gefitinib, in addition to the T790M gatekeeper secondary mutation in EGFR (Engelman et al., 2007; Kobayashi et al., 2005; Yano et al., 2008). HGF secreted from tumor micro-environment has also been suggested as a wide-spread innate resistance mechanism to kinase inhibitors (Straussman at al., 2012; Wilson et al., 2012). Cetuximab, an antibody targeting EGFR, is approved by the FDA for treating head and neck squamous cell carcinoma (HNSCC) and KRAS wild-type EGFR-expressing metastatic colorectal cancer (CRC). However, the objective response rate to Cetuximab in CRC is only 10% to 20% and HGF activation of MET was suggested as a mechanism for primary resistance to Cetuximab in CRC (Liska et al., 2011). Recently, MET amplification was also associated with acquired resistance to Cetuximab in CRC patients (Bardelli at al., 2013).

The goal of this study was to investigate whether activation of MET by stromal HGF can impact Cetuximab efficacy using Cetuximab-sensitive HNSCC and CRC cell lines and whether MET inhibitor INC280 can prevent the HGF effect. In addition, we also tested whether additive/synergistic interaction can be observed by combining Cetuximab with INC280 using this in vitro cell line system.

Methods

### Reagents:

INC280 (Novartis, NVP-INC280) was dissolved in DMSO at 10 mM and stored in aliquots at −20° C. Cetuximab (purchased from Bristol-Myers Squibb) was a 2 mg/ml solution in PBS with no preservatives and stored in aliquots at 4° C. Recombinant HGF (R&D Systems, 294-HG-005/CF) was dissolved in PBS with no preservatives and stored in aliquots at −20° C.

### Cell Culture:

YD-38, CAL-33 and CCK-81 cells were cultured at 37° C. in a 5% C02 incubator using the ATCC media (YD-38, RPMI-1640; CAL-33, DMEM; CCK-81, EMEM) supplemented with 10% FBS (Thermo scientific, SH30071.03). 2 nM L-glutamine (Invitrogen, #25030-081) was also supplemented for CAL-33 media. Cells were passaged twice a week using TryPLE Express (Invitrogen, #12604-013).

### Cell Proliferation Assay:

Cell viability was determined by measuring cellular ATP content using the CellTiter-Glo® (CTG) luminescent cell viability assay (Promega # G7573) according to the manufacturer's protocol. Briefly, various number of cells (6000 for CAL-33, 4200 for YD-38 and 7000 for CCK-81) were seeded in 80 μl growth media per well in clear-bottom 96-well black plates (Costar, #3904) in triplicates. Cells were allowed to attach overnight prior to 72 hours of treatment with indicated compounds (serially-diluted where applicable) and/or 75 ng/ml HGF in a volume of 100 ul for HGF rescue experiment (+20 μl compound or HGF) or 140 ul for Chalice combination experiment (+20 μl compound A+20 μl compound B+20 ul media or HGF). At the end of the drug treatment, 100 ul CTG reagent was added to each well to lyse the cells, and luminescence signals were recorded in the Envision plate reader (Perkin Elmer).

### Method for Calculating the Effect of Combinations:

To evaluate the combination effect in a non-biased way and to identify synergistic effect at all possible concentrations, the combination studies were conducted with a “dose matrix”, where a combination is tested in all possible permutations of serially-diluted Cetuximab and INC280. In all combination assays, compounds were applied simultaneously. This “dose matrix” used in this study is as following: Cetuximab was subjected to a 7 doses 3× serial dilution with the highest dose at 300 nM and the lowest dose at about 137 μM. INC280 was subjected to a 7 doses 3× serial dilution with the highest dose at 1.5 μM and the lowest dose at about 686 μM.

The synergistic interaction was analyzed using Chalice software (CombinatoRx, Cambridge Mass.). Synergy was calculated by comparing a combination's response to those of its single agents, against the drug-with-itself dose-additive reference model and reported as Synergy Score (Lehar et al., 2009).

### Results

1. HGF Rescued the Anti-Proliferation Effect of Cetuximab in HNSCC Cancer Cell Lines:

To test whether HGF can rescue the anti-proliferation effect of Cetuximab, we selected two HNSCC (YD-38 and CAL-33) cell lines that were known to be sensitive to Cetuximab according to previous studies. Using a 3 Day CellTiter-Glo (CTG) luminescent call proliferation assay, 100 nM Cetuximab achieved 70% growth inhibition in YD-38 cells and 36% growth inhibition in CAL-33 cells, respectively (FIG. 1). The lower efficacy of Cetuximab in CAL-33 cells may be partially attributed to the PIK3CA mutation in this cell line (Table 1). Addition of 75 ng/ml HGF (at the same time as Cetuximab treatment) completely rescued the cell growth inhibition by Cetuximab in both cell lines. These data suggest that activation of MET by HGF provided a survival mechanism upon EGFR inhibition in HNSCC cells, which is consistent with the widely recognized notion that MET activation mediates resistance to gefitinib in lung cancer. As a control, HGF alone had no growth-stimulating effect in YD-38 cells, indicating that the rescue of Cetuximab effect by HGF was not a result of stimulating general cell growth. There was a modest growth-promoting effect (˜16%) by HGF alone in CAL-33 cells and the same magnitude of growth increase was also observed in the presence of Cetuximab. Most importantly, the HGF rescue of Cetuximab effect can be completely blocked by co-treatment with 500 nM MET inhibitor INC280 in both cell lines.

2. Cetuximab and INC280 were Synergistic in the Presence of HGF in HNSCC Cancer Cell Lines:

Since INC280 blocked the HGF rescue of Cetuximab effect, we next investigated the combination of INC280 with Cetuximab in suppressing the growth of HNSCC cancer cells in the absence and presence of HGF. In order to evaluate the combination effect in a non-biased way and to identify additive/synergistic effects at al possible concentrations, the study was conducted with a “dose matrix”, where a combination is tested in all possible permutations of serially-diluted Cetuximab and INC280. The “matrix” used in this study was as following: Cetuximab was subjected to a 8 dose 3× serial dilution with the highest dose at 0.3 μM and the lowest dose at about 0.4 nM, and INC280 was subjected to a 8 dose 3× serial dilution with the highest dose at 1.5 μM and the lowest dose at about 2 nM. As expected, Cetuximab single agent showed a potent and concentration-dependent activity of inhibiting proliferation of YD-38 and CAL-33 cells (FIG. 2), and addition of HGF to both cell lines abolished the activity of Cetuximab at nearly all concentrations (FIG. 2). As low as 18 nM INC280 re-sensitized HGF-treated cells to Cetuximab to its original sensitivity level. Calculated using a dose-additive synergistic model, the INC280 and Cetuximab combination in the presence of HGF had synergy score of 12.2 In YD-38 cells and 4.34 in CAL-33 cells, respectively. These scores suggest high synergistic effects by the combination, especially in YD-38 cells. Importantly, the combination synergy was not observed in the absence of HGF and INC280 single agent had no anti-proliferation effect in both cell lines, indicating that INC280 specifically blocked those effects introduced by HGF.

3. HGF Rescued the Anti-Proliferation Effect of Cetuximab in a CRC Cancer Cell Line

Because Cetuximab is also clinically approved for colorectal cancer (CRC), we performed HGF rescue experiments in CCK-81 CRC cells that are known to be sensitive to Cetuximab to extend our findings. In a 3 Day CTG luminescent cell proliferation assay, 100 nM Cetuximab achieved 67% growth inhibition in CCK-81 cells (FIG. 3). Similarly, addition of 75 ng/ml HGF together with Cetuximab completely rescued the cell growth inhibition by Cetuximab in CCK-81 cells, while HGF alone had only modest growth-stimulating effect (˜19%). Again, the HGF rescue of Cetuximab effect can be fully blocked by co-treatment with 500 nM MET inhibitor INC280.

4. Cetuximab and INC280 were Synergistic in the Presence of HGF in a CRC Cancer Cell Line

The activity of combining INC280 with Cetuximab in suppressing growth of CCK-81 cells in the absence and presence of HGF in the above mentioned “dose matrix” experiment were also investigated. Cetuximab single agent displayed a potent and concentration-dependent activity of inhibiting proliferation of CCK-81 cells and addition of HGF abolished the activity of Cetuximab at nearly all concentrations (FIG. 4). Again, as low as 18 nM INC280 re-sensitized HGF-treated cells to Cetuximab to its original sensitivity level. Judged from synergy scores, the INC280 and Cetuximab combination was highly synergistic in the presence of HGF with synergy score of 14 but not synergistic at all in the absence of HGF with synergy score of 0.12. Modest growth inhibition by INC280 single agent at 1.5 μM (˜20%) was observed in the presence of HGF, which may be a result of blocking the slight growth-stimulating effect by HGF in CCK-81 cells (˜20%) as seen in FIG. 3.

TABLE 1
Genetic background of cell lines used in the study

| | | EGFR- | EGFR- | MET- | MET- | HGF- | |
|---|---|---|---|---|---|---|---|
| Cell Line | Lineage | CN | MAS5 | CN | MAS5 | MAS5 | Mutations |
| YD-38 | HNSCC | 3.3 | 5962.6 | 2.2 | 18452.2 | 12.9 | |
| CAL-33 | HNSCC | 4.0 | 8883.3 | 2.0 | 10154.3 | 0.9 | PIK3CA |
| CCK-81 | CRC | 2.1 | 248.6 | 2.2 | 5124.9 | 1.0 | |

### CONCLUSION AND DISCUSSION

In this study, we found that hepatocyte growth factor (HGF) rescued both HNSCC and CRC cells from the effects of EGFR inhibition by Cetuximab on proliferation. INC280, a highly selective inhibitor of the HGF receptor MET, abolished the effects of HGF and re-sensitized HNSCC and CRC Cells to Cetuximab. The effect of INC280 was seen at as low as 18 nM and this concentration in tumors is expected to be clinically achievable. In addition, the INC280 and Cetuximab combination was highly synergistic in the presence of HGF with synergy scores ranging from 4.3 to 14.0, using a well-accepted dose-additive synergistic model. The combination synergy was not observed in the absence of HGF and INC280 as a single agent had little or no anti-proliferation effect in HNSCC and CRC cells regardless of HGF addition.

The HNSCC and CRC cell line models we selected are sensitive to Cetuximab, probably due to high expression of either EGFR (Table 1) or its various ligands. The HGF expression levels in those cell lines are extremely low with HGF MAS5<13, which enabled us to achieve MET activation by adding exogenous HGF. In addition, there is no MET amplification in any of those three cell lines by copy number analysis (Table 1). MET expression is generally high in those three cell lines with YD-38 having the highest MET MAS5 of 18452.2. As a comparison, the median MET expression of 32 HNSCC cancer cell lines in our CCLE collection (Barretina at al., 2012) is 10154.3 and the MET expression of a MET-amplified gastric cell line MKN-45 is 29714.7, which may be approaching the assay limit. Therefore, the MET expression levels in our cell line models are likely to be representative of their lineages and well below the level seen in MET-amplified models.

Because mouse Hgf does not activate human MET, it is not straightforward to test the HGF rescue of Cetuximab effect in xenograft models unless using HGF transgenic mice. Alternatively, MET-amplified HNSCC or CRC models can be utilized to mimic the HGF rescue effect for testing INC280 and Cetuximab combination in vivo. In conclusion, our data provide a rationale for combining Cetuximab with INC280 in the clinic to potentially both overcome the primary resistance and prevent the acquired resistance to Cetuximab mediated by various types of MET activation such as HGF overexpression or MET amplification in HNSCC and CRC tumors.

### Example 2: A Phase Ib, Open-Label, Multicenter, Dose Escalation and Expansion Study, to Evaluate the Safety, Pharmacokinetics and Activity of INC280 in Combination with Cetuximab in c-MET Positive CRC and HNSCC Patients Who have Progressed after Anti-EGFR Monoclonal Antibody Therapy

### LIST OF ABBREVIATIONS

AE(s) Adverse Event(s)
ALT/GPT Alanine aminotransferase/glutamic pyruvic transaminase
ANC Absolute neutrophil count
AST/GOT Aspartate aminotransferase/glutamic oxaloacetic transaminase
ATC Anatomical Therapeutic Chemical
ATP Adenosine Triphosphate
AUC Area under the concentration-time curve
Bid bis in diem/twice a day
BCRP Breast cancer resistant protein
BLRM Bayesian Logistic Regression Model
BOR Beet Overall Response
BUN Blood urea nitrogen
CBC Complete blood count
Cmax Maximum concentration
CNS Central nervous system
CRC/mCRC Colorectal cancer/metastatic CRC
CrCl Creatinine clearance
CR Complete response
CRO Contract Research Organization
CSR Clinical study report
CT Computed tomography
CTCAE Common terminology criteria for adverse events
CYP Cytochrome P450
DDI Drug-drug interaction
DDS Dose determining set
DLT(s) Dose Limiting Toxicity(ies)
DMC Data Monitoring Committee
DS&E Drug Safety and Epidemiology
eCRF Electronic Case Report/Record Form
ECG(s) Electrocardiogram(s)
ECHO Echocardiogram
ECOG Eastern Cooperative Oncology Group
EGF Epidermal Growth Factor
EGFR/EGFRi Epidermal growth factor receptor/EGFR inhibitor
EMEA European Medicines Evaluation Agency
EOT End of Treatment
EWOC Escalation with overdose control
FAS Full Analysis Set
FDA Food and Drug Administration
FDG-PET Fluorodeoxyglucose Positron Emission Tomography
hCG Human chorionic gonadotropin
HGF Hepatocyte Growth Factor
HIV Human Immunodeficiency Virus
HNSCC Head and Neck Squamous Cell Carcinoma
HR Hazard Ratio
i.v. Intravenous(ly)
IB Investigator's Brochure
ICF Informed Consent Form
ICH International Conference on Harmonization
IEC(s) Independent Ethics Committee(s)
INR International Normalized Ratio
IRB(s) Institutional Review Board(s)
IUD Intrauterine Device
IUS Intrauterine System
KRAS V-Ki-ras2 Kirsten rat sarcoma viral oncogene homolog
LLN Lower Limit of Normal
LLOQ Lower Limit of Quantitation
LVEF Left ventricular ejection fraction
MRI Magnetic Resonance imaging
MTD Maximum Tolerated Dose
MUGA Multigated Acquisition
NaF PET Sodium fluoride positron emission tomography
NCCN National Comprehensive Cancer Network
NOAEL No Adverse Effect Level
NRAS Neuroblastoma RAS viral oncogene homolog
NSCLC Non-Small Cell Lung Cancer
ORR Overall Response Rate
OS Overall Survival
PAS Pharmacokinetic Analysis Set
P-gp Permeability glycoprotein
PD Pharmecodynamic
PFS Progression Free Survival
PK Pharmacokinetic
PK/PD Pharmacokinetic/Pharmacodynamic
PR Partial Response
PT Prothrombin time
qd once a day
qwk weedy
RAP Report and Analysis Plan (a regulatory document which provides evidence of preplanned analyses)
RAS Rat sarcoma viral oncogene homologue
RDE Recommended Dose for Expansion
REB Research Ethics Board
RECIST Response Evaluation Criteria in Solid Tumors
RP2D Recommended Phase two Dose
RR Response Rate
SAE(s) Serious Adverse Event(s)
SDH Sorbiol Dehydrogenase
TLS Tumor Lysis Syndrome
TSH Thyroid Stimulating Hormone
ULN Upper Limit of Normal
US United States
WBC White Blood Cells
WT Wild Type

### Glossary of Term

| | |
|---|---|
| Assessment | A procedure used to generate data required by the study |
| Cohort | A group of newly enrolled patients treated at a specific dose and regimen at the same |
| | time |
| Cycle | Number and timing or recommended repetitions of therapy are usually expressed as |
| | number of days (e.g.: q28 days) |
| Dose level | The dose of drugs given to the patient |
| Enrollment | Point/time of patient entry into the study; the point at which informed consent must be |
| | obtained (i.e. prior to starting any of the procedures described in the protocol) |
| Period | A subdivision of the study timeline; divides stages into smaller functional segments such |
| | as screening, treatment, follow-up etc. |
| Stage in cancer | The extent of a cancer in the body. Staging is usually based on the size of the tumor, |
| | whether lymph nodes contain cancer, and whether the cancer has spread from the |
| | original site to other parts of the body |
| Stage related to study | A major subdivision of the study timeline; begins and ends with major study milestones |
| timeline | such as enrollment, completion of treatment, etc. |
| Study evaluation | Point/time which marks the end of study for an individual patient. Assessment of survival |
| completion | continues beyond the study evaluation completion |
| Study treatment | Includes any drug or combination of drugs administered to the patient (subject) as part of |
| | the required study procedures |
| Study treatment | Point/time when patient permanently stops taking study treatment for any reason; may or |
| discontinuation | may not also be the point/time of premature patient withdrawal |
| Subject Number (Subject | A unique identifying number assigned to each patient who enrolls in the study |
| No.) | |
| Supportive treatment | Refers to any treatment required by the exposure to a study treatment, e.g. |
| | premedication of vitamin supplementation and corticosteroid for pemetrexed disodium |
| Variable | Identifier used in the data analysis; derived directly or indirectly from data collected using |
| | specified assessments at specified timepoints |

A phase Ib, open-label, multicenter, dose escalation and expansion study, to evaluate the safety, pharmacokinetics and activity of INC280 in combination with cetuximab in c-MET positive CRC and HNSCC patients who have progressed after anti-EGFR monoclonal antibody therapy is being planned.

In HNSCC, c-MET amplification has been observed in 13% of cases (Seiwert et al 2009), but currently no clinical data have shown a correlation between c-MET amplification and acquired resistance to anti-EGFR antibody therapies.

In order to explore the hypothesis that Inhibition of c-MET could overcome the acquired resistance to EGFR Inhibitors, this study will combine the c-MET Inhibitor INC280 with the EGFR Inhibitor, cetuximab, in mCRC and HNSCC patients whose tumors have become resistant to anti-EGFR treatment through activation of the MET receptor.

A newly obtained tumor biopsy must be taken at the time of cetuximab or panitumumab progression, during molecular pre-screening, and is a mandatory criterion for the inclusion of patients in the expansion part of the study. This tumor sample will enable a more accurate assessment of the current biological phenotype of the tumor. Furthermore, the availability of previously obtained tumor material will allow a comprehensive understanding of the genetic alterations by comparing the newly obtained biopsy with the initial genetic profile of the tumor in a large and controlled patient population.

### Rationale for the Study Design

This is an open label, phase Ib dose escalation study followed by an expansion part of INC280 in combination with cetuximab in adult patients with c-MET positive (as defined by c-MET IHC Intensity score +2 In ≥50% of tumor cells and MET gene copy number ≥5 by FISH or IHC Intensity score +3 In ≥50% of tumor cells. Patients with MET gene copy number ≥5 and unknown c-MET IHC results or c-MET mutation can be enrolled in the study following discussion and agreement with Novartis.) mCRC (K/NRAS-WT status) and HNSCC whose disease progressed after cetuximab and/or panitumumab treatment.

The purpose of the dose escalation part of the study is to determine the MTD and/or Recommended Dose for Expansion (RDE) of INC280 in combination with cetuximab. In addition to evaluating the safety, tolerability and the PK, this study Is designed to provide a preliminary assessment of the efficacy of this combination. The dose escalation part will be guided by a Bayesian Logistic Regression Model (BLRM).

This open-label dose escalation study design using a BLRM is a well-established method to estimate the MTD and/or RDE in cancer patients. The adaptive BLRM will be guided by the escalation with overdose control (EWOC) principle to control the risk of DLT in future patients on study. The use of Bayesian response adaptive models for small datasets has been accepted by the European Medicines Evaluation Agency (EMEA) (“Guideline on clinical trials in small populations”, Feb. 1, 2007) and endorsed by numerous publications (Babb 1998 et al, Neuenschwander et al 2008, Neuenschwander et al 2010), and its development and appropriate use is one aspect of the Food and Drug Administration's (FDA) Critical Path Initiative.

The decisions on new dose levels are made by the Investigators and Novartis study personnel and will be based upon the recommendations made by the BLRM, patient tolerability and safety, PK, pharmacodynamic (PD) and efficacy Information available at the time of the decision.

Once the MTD and/or RDE has been established, additional mCRC and HNSCC patients who have progressed on cetuximab or panitumumab treatment will be enrolled in two expansion groups to further assess the anti-tumor activity of the combination. The expansion part will continue to assess the safety and tolerability of INC280 and cetuximab at the MTD/RDE.

### Rationale for Dose and Regimen Selection

The selection of the oral dosing schedule and the initial starting dose for the dose escalation part of INC280 using the tablet formulation are based on the currently available safety, PK, PD and preliminary efficacy data from the completed and ongoing clinical studies with INC280 (in capsule formulation) and upon the clinical experience of a relative bioavailability study, which compared the two formulations in healthy volunteers.

The starting dose for INC280 tablets selected for this study is 150 mg bid on a continuous dosing schedule based on the AUC ratios and available strengths of the tablet formulation.

The selected starting dose of INC280 on a continuous twice daily schedule in combination with cetuximab is supported by the risk assessment (EWOC) within the BLRM derived from single-agent INC280 dose-DLT data and predicted interaction with cetuximab.

The fixed dose of cetuximab, 400 mg/m2 Initial dose and subsequent weekly doses of 250 mg/m2 follows the recommended dosing for mCRC and HNSCC patients according to the cetuximab label. No drug-drug Interaction (DDI) at the PK level is expected between INC280 and cetuximab.

### Rationale for Choice of Combination Drugs

This study is designed to explore if the combination of the c-MET inhibitor, INC280, and the EGFR inhibitor, cetuximab, will provide clinical benefit to mCRC and HNSCC patients whose tumors have become resistant to anti-EGFR treatment through activation of the MET receptor by overcoming this resistance.

### Study Design

### Description of Study Design

This is a multi-center, open-label, dose finding, Phase Ib dose escalation study to estimate the MTD and/or RDE for the combination of INC280 and cetuximab, followed by an expansion part to assess the clinical efficacy and to further assess the safety of the combination in c-MET positive (refer to Section 5.2 for detailed definition) mCRC and HNSCC patients who progress following cetuximab or panitumumab treatment.

The phase Ib dose escalation part of the study will be conducted in adult c-MET positive and K/NRAS WT mCRC and c-MET positive HNSCC patients. Cohorts of patients will be treated with the combination until the MTD and/or RDE of the combination is Identified. Approximately 20 patients will be enrolled in the dose escalation part.

Following MTD and/or RDE declaration, patients will be enrolled in 2 expansion groups. Group 1 will consist of approximately 20 c-MET positive and K/NRAS WT mCRC patients who have progressed after treatment with an EGFR inhibitor (EGFRi) (cetuximab or panitumumab) and have received at least one previous line of treatment for their metastatic disease. Group 2 will consist of approximately 20 c-MET positive HNSCC patients who have progressed after treatment with cetuximab and have received at least one previous line of treatment for their metastatic disease.

A patient may enroll on an optional companion protocol to study the mechanisms of resistance to INC280 and cetuximab. Patients who agree to participate in the companion study will provide samples for analysis of their cancer at study entry and again upon the development of resistance.

Molecular Pre-Screening

To enter the screening period of the study, patients must have evidence of c-MET positivity. For the mCRC patients additional written documentation of K/NRAS-WT status is required.

Patients to be enrolled in the expansion part of the study must sign the molecular pre-screening consent to allow for the mandatory collection of a newly obtained tumor sample. In addition, patients to be enrolled in the dose escalation part will sign the molecular pre-screening consent if previously obtained local documentation for c-MET positivity and K/NRAS WT status (for mCRC patients) is not available.

Screening Period

The screening period begins once the patient has signed the study Inform consent. Patients will be evaluated against study inclusion and exclusion criteria and safety assessments.

Treatment Period

The treatment period will begin on Cycle 1 Day 1. The study treatment will be administered during 28-days cycles. Patients will be treated until progression of disease, development of unacceptable toxicity, withdrawal of Informed consent or death, whichever occurs first

Safety Follow-Up Period

Patients will be followed up for safety evaluations for 30 days after the last administration of study treatment.

Disease Progression and Survival Follow-Up Period (Expansion Part Only)

Patients enrolled in the expansion part of the study who discontinue study treatment for any reason other than disease progression will be followed up for progression of disease. In addition, patients in the expansion part will be followed for survival.

### Timing of Interim Analyses and Design Adaptations

No formal interim analyses are planned for the study. However, the dose-escalation design foresees that decisions based on the current data are taken before the end of the study. In addition, data from patients in the expansion groups will be reviewed on an ongoing basis to monitor the safety and tolerability of the MTD/RDE in that part of the study.

### Definition of End of the Study

End of study will be upon completion of the survival follow-up period of the last patient treated with the combination of INC280 and cetuximab, or when the study is terminated early.

Completion of the survival follow-up period is once the last patient in the dose expansion pert has died or has been followed for survival up to 6 months after the last dose of study treatment, whichever occurs first Completion of the survival follow-up period could also be considered if >80% of patients have died or are lost to follow-up.

Patient Population

The patient population of the study consists of adult patients with K/NRAS WT and c-MET positive mCRC and c-MET positive recurrent/metastatic HNSCC who have received at least one previous line of treatment for the metastatic disease. The last treatment should include an anti-EGFR antibody (cetuximab/panitumumab or only cetuximab for HNSCC). For the expansion part of the study, documentation of clinical benefit and subsequent progression of disease while on cetuximab/panitumumab (or only cetuximab for HNSCC patients) treatment is required.

The investigator or designee must ensure that only patients who meet all the following inclusion and none of the exclusion criteria are offered treatment in the study. All data for the inclusion and exclusion criteria must be verifiable in the patient's source documents. Patients enrolled in this study are not permitted to participate in additional parallel Investigational drug or device studies. Patients who have completed the study may not be re-enrolled for a second course of treatment.

Inclusion Criteria

Patients eligible for Inclusion in this study have to meet all of the following criteria:

1. Male or female patients aged ≥18 years

2. Histological or cytological confirmation of mCRC or HNSCC. The availability of a representative, most recent, previously obtained tumor sample with a corresponding pathology report Is mandatory to be collected at molecular pre-screening/screening for the analyses described in the protocol. In exceptional situations after discussion with Novartis, only the newly obtained tumor sample will be sufficient.

3. Written documentation of c-MET positivity as defined by c-MET IHC Intensity score +2 in ≥50% of tumor cells and MET gene copy number ≥5 by FISH or IHC intensity score +3 in ≥50% of tumor cells and K/NRAS-WT status (KRAS and NRAS, exons 2, 3 and 4) for mCRC patients only. Patients with MET gene copy number ≥5 (by FISH) and unknown c-MET IHC results or c-MET mutation can be enrolled in the study following discussion and agreement with Novartis. The analysis may be performed locally or through a Novartis designated central laboratory

For Dose Escalation part: Analysis can be performed on a newly obtained or the most recent previously obtained tumor sample available.

For Expansion part: The analysis will be performed only on a newly obtained tumor sample.

If a tumor sample was collected within 3 months prior to start of treatment on this study and after the most recent anti-neoplastic regimen which must have contained cetuximab or panitumumab, then this tumor sample will be acceptable for enrollment in this case a newly obtained tumor sample is not required.

Alternatively, and in exceptional situations after discussion with Novartis, a previously obtained tumor sample with a corresponding pathology report would be allowed.

4. mCRC patients in dose escalation part: At least one previous line of treatment for the metastatic disease and the last treatment must have included cetuximab or panitumumab as a single agent or in combination with chemotherapy. For patients in the expansion part, additional documentation of clinical benefit (complete or partial response or stable disease) and subsequent progression of disease while on continuous cetuximab or panitumumab as the most recent line of treatment is required.

5. HNSCC patients in dose escalation part: At least one previous line of treatment for the recurrent or metastatic disease and the last treatment must have Included cetuximab as a single agent or in combination with chemotherapy. For patients in the expansion part, additional documentation of clinical benefit (complete or partial response or stable disease) and subsequent progression of disease while on continuous cetuximab as the most recent line of treatment is required.

6. At least one tumor lesion meeting measurable disease criteria as per RECIST v1.1. Lesions in previously irradiated areas or those that have received other locoregional therapies (i.e. percutaneous ablation) should not be considered measurable unless there is clear documented evidence of progression of the lesion since therapy.

7. Eastern cooperative oncology group (ECOG) performance status ≥2

8. Able to understand and voluntarily sign the Informed consent form and ability to comply with the study visit schedule and the other protocol requirements, including the collection of a newly obtained tumor sample. Written Informed consent must be obtained prior to any molecular pre-screening and screening procedures.

Exclusion Criteria

Patients eligible for this study must not meet any of the following criteria:

1. Prior treatment with c-MET/HGF Inhibitors.

2. Known history of severe reactions (except for G3 rash and G3 hypomagnesaemia) to cetuximab or panitumumab

3. Symptomatic CNS metastases which are neurologically unstable or requiring increasing doses of steroids to control their CNS disease. Note: Patients with controlled CNS metastases may participate in this trial. The patient must have completed radiotherapy or surgery for CNS metastases >4 weeks prior to starting study treatment. Patients must be neurologically stable, having no new neurologic deficits on clinical examination, and no new findings on CNS imaging. If patients require steroids for management of CNS metastases, they must have been on a stable dose of steroids for two weeks preceding study entry

4. Significant or uncontrolled cardiovascular disease (e.g., uncontrolled hypertension, peripheral vascular disease, congestive heart failure, cardiac arrhythmia, or acute coronary syndrome) within 6 months of starting study treatment or myocardial Infarction within 12 months of starting study treatment

5. Have any of the following laboratory values at screening/baseline:
Absolute neutrophil count (ANC)<1,500/mm3 [1.5×109/L]
Platelets <75,000/mm3 [75×109/L]
Hemoglobin <9.0 g/dL
Serum creatinine >1.5× upper limit of normal (ULN) and/or calculated or directly measured creatinine clearance (CrCl)≤45 mL/min
Serum total bilirubin >2 mg/dL (or >1.5×ULN if liver metastases are present; or total bilirubin >3.0×ULN with direct bilirubin >normal range in patients with well documented Gilbert's Syndrome, which is defined as presence of several episodes of unconjugated hyperbilirubinemia with normal results from complete blood count (CBC) (including normal reticulocyte count and blood smear), normal liver function test results, and absence of other contributing disease processes at the time of diagnosis)
AST/SGOT or ALT/SGPT>2.5×ULN, or >5.0×ULN If liver metastases are present
Hypomagnesaemia≥CTCAE Grade 1 (lower limit of normal (LLN)-1.2 mg/dL or LLN-0.5 mmol/L). Replacement therapy is allowed.
Serum albumin <2.8 g/dL
Asymptomatic serum amylase and lipase >CTCAE Grade 2 (1.5-2.0×ULN)
Serum amylase or serum lipase CTCAE grade ≥1 with signs and/or symptoms suggesting pancreatitis or pancreatic Injury (e.g. elevated P-amylase, abnormal Imaging findings of pancreas, etc)
International normalized ratio (INR)>1.5×ULN or prothrombin time (PT)>6 seconds above control

6. Impairment of gastrointestinal (GI) function or GI disease that may significantly alter the absorption of oral INC280 (e.g., ulcerative diseases, uncontrolled nausea, vomiting, diarrhea, malabsorption syndrome, small bowel resection)

7. Previous or concurrent malignancy. Exceptions: adequately treated basal cell or squamous cell skin cancer; In situ carcinoma of the cervix, treated curatively and without evidence of recurrence for at least 3 years prior to study entry; or other solid tumor treated curatively, and without evidence of recurrence for at least 3 years prior to study entry

8. History of thromboembolic or cerebrovascular events within the last 6 months, including transient ischemic attack, cerebrovascular accident, deep vein thrombosis, or pulmonary embolism

9. Prior radiation therapy (that includes >30% of the bone marrow reserve) with exception of palliative radiotherapy, chemotherapy, biological therapy (excluding cetuximab and panitumumab) within ≤4 weeks (6 weeks for nitrosourea, mitomycin-C), or treatment with continuous or Intermittent small molecule therapeutics or investigational agents within 5 half-lives of the agent (or ≤4 weeks when half-life is unknown) prior to starting study drug or not recovery to grade ≤1 from the side effects of such therapy (except alopecia and neuropathy).

10. Major surgical procedure, open biopsy, or significant traumatic injury within 4 weeks prior to starting study treatment or patients who have not recovered from the side effects of such procedure

11. Active bleeding within 4 weeks prior to screening visit including variceal bleeding (esophageal varices should be treated according to standard practice e.g. ligation or banding and procedure completed 4 weeks prior to screening visit)

12. Clinically significant third space fluid accumulation (i.e., ascites or pleural effusion requiring fluid removal despite the use of diuretics or associated with shortness of breath)

13. History of acute or chronic pancreatitis or any risk factors that may increase the risk of pancreatitis

14. Currently receiving increasing or chronic treatment (>5 days) with corticosteroids or another immunosuppressive agent Note: Single doses, topical applications (e.g., for rash), inhaled sprays (e.g., for obstructive airway diseases), eye drops or local injections (e.g., intraarticular) are allowed. Patients who are on a stable or decreasing low dose of corticosteroid treatment (e.g., dexamethasone not exceeding 4 mg/day or other corticosteroids equivalent dose) for at least 5 days before start of study treatment are eligible

15. Known history of human immunodeficiency virus (HIV) seropositivity. HIV testing is not required as part of this study

16. Receiving treatment with medications that are known strong inhibitors or inducers of CYP3A4, and cannot be discontinued 7 days prior to the start of INC280 treatment and during the course of the study (refer to Appendix 3)

17. Receiving treatment with medications that are known CYP3A4, CYP1A2, CYP2C8, CYP2C9 or CYP2C19 substrates with narrow therapeutic index, and cannot be discontinued before start of study treatment (refer to Appendix 3)

18. Receiving treatment with long acting proton pump inhibitors, and cannot be discontinued 3 days prior to the start of INC280 treatment (refer to Appendix 3)

19. Feeding tube dependence

20. Any other condition that would, in the Investigator's judgment, contraindicate patient's participation in the clinical study due to safety concerns or compliance with clinical study procedures, e.g., infection/inflammation, intestinal obstruction, social/psychological issues etc.

21. Pregnant or nursing (lactating) women, where pregnancy is defined as the state of a female after conception and until the termination of gestation, confirmed by a positive hCG laboratory test.

22. Women of child-bearing potential, defined as all women physiologically capable of becoming pregnant, unless they are using highly effective methods of contraception during dosing and for at least 4 weeks after permanently discontinuing study treatment Highly effective contraception methods Include:
Total abstinence (when this is in line with the preferred and usual lifestyle of the subject. Periodic abstinence (e.g., calendar, ovulation, symptothermal, post-ovulation methods) and withdrawal are not acceptable methods of contraception
Female sterilization (have had surgical bilateral oophorectomy with or without hysterectomy) or tubal ligation at least six weeks before taking study treatment. In case of oophorectomy alone, only when the reproductive status of the woman has been confirmed by follow up hormone level assessment
Male sterilization (at least 6 months prior to screening). For female subjects on the study the vasectomized male partner should be the sole partner for that subject.
Combination of any two of the following (a+b or a+c, or b+c):

a. Use of oral, injected or implanted hormonal methods of contraception or other

forms of hormonal contraception that have comparable efficacy (failure rate <1%), for example hormone vaginal ring or transdermal hormone contraception.

b. Placement of an intrauterine device (IUD) or intrauterine system (IUS)

c. Barrier methods of contraception: Condom or Occlusive cap (diaphragm or cervical/vault caps) with spermicidal foam/gel/film/cream/vaginal suppository

In case of use of oral contraception women should have been stable on the same pill for a minimum of 3 months before taking study treatment.

Women are considered post-menopausal and not of child bearing potential if they have had 12 months of natural (spontaneous) amenorrhea with an appropriate clinical profile (e.g. age appropriate, history of vasomotor symptoms) or have had surgical bilateral oophorectomy (with or without hysterectomy) or tubal ligation at least six weeks ago. In the case of oophorectomy alone, only when the reproductive status of the woman has been confirmed by follow up hormone level assessment is she considered not of child bearing potential.

23. Sexually active males unless they use a condom during Intercourse while taking drug and for at least 4 weeks after stopping study treatment and should not father a child in this period. A condom is required to be used also by vasectomized men in order to prevent delivery of the drug via seminal fluid.

Dosing Regimen

Patients will be assigned to receive the combination of INC280 on a continuous bid dosing regimen and cetuximab every week (qwk) (Table 2).

TABLE 2
Dose and treatment schedule

| | | | Frequency |
|---|---|---|---|
| Study | Pharmaceutical form and | | and/or |
| drugs | route of administration | Dose | Regimen |
| INC280 | tablet for oral use: | as assigned during | bid |
| | whole tablets or | dose escalation and | |
| | suspension prepared from | the declared RDE | |
| | crushed tablets | for the | |
| | | expansion part | |
| Cetuximab | intravenous infusion | 400 mg/m² initial | qwk |
| | | infusion | |
| | | 250 mg/m² subsequent | |
| | | infusions | |

INC280 Administration

INC280 will be administered as a flat dose of mg/day and not Individually adjusted by body weight or body surface area.

INC280 will be administered orally and on a continuous bid dosing schedule.

Patients should be instructed to take their doses at approximately the same time each day. The second (evening) dose should be taken 12 (±2) hours after the morning dose. INC280 should be administered in the fasted state, at least one hour before or two hours after a meal. During fasting period, patients can freely drink water.

Patients must avoid consumption of Seville orange (and Juice), grapefruit or grapefruit juice, grapefruit hybrids, pummelos and star citrus fruits at least 7 days prior to the first dose of study drug and during the entire study treatment period due to potential CYP3A Interaction. Regular orange juice (Citrus sinensis) Is allowed.

Patients should be instructed to swallow the tablets whole and not to chew them. For patients with swallowing dysfunction, the INC280 tablets can be administered as drinkable suspension by crushing the tablets and suspending them in water. Investigators and patients will receive detailed Instructions on how to prepare the drinkable suspension. The drinkable suspension is not permitted for administration into feeding tubes.

If a significant difference in PK profile is observed between the tablets taken whole and crushed, which is leading to differences in the RDE, then future patients to be recruited in the dose escalation and/or expansion part will be required to take the tablet whole and additional patients with swallowing dysfunction will not be recruited to the study.

On the days when PK blood samples are to be collected, patients will be Instructed to hold their dose of study drug until arrival at the study center. The administration of study drug will be supervised by the study personnel and the time of administration will be recorded. The same dietary restrictions for dosing will be in place on days with PK sampling (INC280 should be administered in the fasted state, at least one hour before or two hours after a meal).

If vomiting occurs, no attempt should be made to replace the vomited dose. If any episodes of vomiting occurred within the first 4 hours of study drug dosing, on PK sampling days of Cycle 1, exact time of vomiting should be recorded on the appropriate eCRF besides the AE eCRF.

A missed dose is defined as any time point when a patient forgets to take study drug within 4 hours after the planned time of dosing or if a patient forgets to take his/her dose for that day. In such cases, the dose should be omitted and the patient should continue treatment with the next scheduled dose.

Cetuximab Administration

Cetuximab will be administered intravenously weekly at the study site on Days 1, 8, 15 and 22 (+3 days) of the 28-day cycle, as per cetuximab label instructions. Pre-medication, if required, should be administered following Institutional standards 30 minutes prior to cetuximab infusion. The cetuximab Initial dose (Cycle 1 Day 1) is 400 mg/m2 administered as a 120-minute intravenous Infusion followed by 250 mg/m2 weekly doses Infused over 60 minutes (second Infusion onward). The Infusion rate should not exceed 10 mg/min. Close monitoring is required during the infusion and for at least 1 hr after the end of the infusion.

If an infusion reaction occurs while cetuximab is being administered, the infusion should be stopped Immediately and the patient should be closely monitored and treated in line with Institutional standards. Upon resolution of symptoms, for Grade 1 or 2 infusion reactions and non-serious Grade 3 infusion reactions, reduce the Infusion rate by at least 50%. For patients with serious infusion reactions, cetuximab treatment must be Immediately and permanently discontinued.

Sequence of INC280 and Cetuximab Administration

Pre-medication that has the potential to alter the pH of the upper GI tract may alter the solubility of INC280 and hence its bioavailability. These agents Include, but are not limited to, H2-antagonists (e.g., ranitidine) and antacids. Therefore, oral dosing of INC280 will be administered prior to cetuximab and its premedication. This sequence will also allow for consistent timing of the INC280 morning dose administration.

A minimum of 1 hour must pass from the time of INC280 administration to the administration of cetuximab premedication (if required). Cetuximab infusion is recommended 30 minutes post-premedication (i.e. 1.5 hour post-INC280 intake).

Ancillary Treatments

Pre-medication for cetuximab should be administered as per standard institutional guidelines and/or as described in the locally applicable cetuximab label.

Treatment Duration

Patients will be treated with the study treatment until patient experiences unacceptable toxicity, disease progression, death, withdraws prematurely and/or upon withdrawal of consent, whichever occurs first.

Dose Escalation Guidelines

Starting Dose Rationale

The starting dose for INC280 is 150 mg bid administered continuously in combination with a fixed dose of cetuximab of 400 mg/m2 as the initial dose (C1D1) and 250 mg/m2 as subsequent weekly doses in 28-day cycles. Refer to Section 2.3 for the rational on the selection of the starting dose.

Taking into consideration all Information currently available about the dose-DLT relationships of INC280 and cetuximab as single agents and the uncertainty about the toxicity of the combination, the prior distribution of DLT rates derived from the BLRM indicates that the proposed starting dose combination meets the EWOC.

Provisional Dose Levels

Table 3 describes the starting dose and the dose levels of INC280 that may be evaluated during this study. With the exception of starting dose level 1, actual dose levels will be determined following a discussion with the participating Investigators during the dose escalation teleconferences. Dose escalation will continue until the MTD/RDE is reached.

The dose for cetuximab will be fixed for all dose escalation cohorts.

TABLE 3
Provisional dose levels for INC280

| | | Proposed INC280 |
|---|---|---|
| Dose level | Proposed INC280 mg* | Total Daily Dose |
| −1** | 100 | 200 mg |
| 1/starting dose | 150 | 300 mg |
| 2 | 200 | 400 mg |

*It is possible for additional and/or intermediate dose levels to be added during the course of the study Cohorts may be added at any dose level below the MTD/RDE in order to better understand safety or PK. In addition, dose levels may be explored in parallel.
**Dose level −1 represents treatment dose for patients requiring a dose reduction from the starting dose level. No dose reduction below dose level −1 is permitted for this study.

At all decision time points, the adaptive BLRM permits alterations in the dose increments based on the observed DLTs.

Guidelines for Dose Escalation and Determination of MTD/RDE

For the purposes of dose escalation decisions, each cohort will consist of 3 to 6 newly enrolled patients who will be treated at the specified dose level. The first cohort will be treated with the starting dose of INC280 as shown in Table 3 in combination with the fixed dose for cetuximab.

Patients must complete a minimum of 1 cycle (28 days) of treatment with the minimum safety evaluation and drug exposure or have had a DLT within the first cycle of treatment to be considered evaluable for dose escalation decisions. Dose escalation decisions will occur when the cohort of patients has met these criteria.

Dose escalation decisions will be made by investigators and Novartis study personnel. Decisions will be based on a synthesis of all relevant data available from all dose levels evaluated in the ongoing study including safety information, DLTs, all CTCAE Grade ≥2 toxicity data during Cycle 1, PK and PD data from evaluable patients. The recommended dose for the next cohort of subjects will be guided by the BLRM with EWOC principle.

The adaptive Bayesian methodology provides an estimate of all dose levels of INC280 in combination with cetuximab that do not exceed the MTD and incorporates al DLT information at all dose levels for this estimation. In general, the next dose will have the highest chance that the DLT rate will fall in the target Interval [16-35%) and will always satisfy the EWOC principle. In all cases, the dose for the next cohort will not exceed a 100% increase from the previous dose. Smaller increases in dose may be recommended by the Investigators and Novartis upon consideration of all of the available clinical data.

If 2 patients in a previously untested dose level experience a DLT, enrollment to that cohort will stop, the BLRM will be updated and the next cohort will be opened at the next lower dose level or an intermediate dose level (Appendix 2) that satisfies the EWOC criteria. However, if 2 patients in a new cohort at a previously tested dose level experience a DLT (e.g., a total of 8 patients are treated on this dose level with 2 DLT observed), further enrollment to that cohort will stop, the BLRM will be updated with this new information and re-evaluation of the available safety, PK and PD data will occur. By incorporating information gained at the preceding dose cohorts, additional patients may be enrolled Into the current dose cohort only if the dose still meets the EWOC criteria and as agreed by Investigators and Novartis personnel. Alternatively, if recruitment to the same cohort may not resume, a new cohort of patients may be recruited to a lower dose as agreed by Investigators and Novartis personnel and if the BLRM predicts that the risk for this lower dose combination to exceed the MTD remains below 25% (EWOC). Re-escalation may then occur if data in subsequent cohorts supports this (EWOC criteria are satisfied) and Investigators and Novartis personnel agree.

Dose escalation will continue until identification of the MTD or a suitable lower dose for expansion. This will occur when the following conditions are met:

1. at least 6 patients have been treated at this dose

2. this dose satisfies one of the following conditions:

a. the posterior probability of targeted toxicity at this dose exceeds 50% and is the highest among potential doses, or

b. minimum of 12 patients have already been treated on the trial.

3. It is the dose recommended for patients, either per the model or by review of all clinical data by Novartis and Investigators in a dose-escalation teleconference, see Section 6.2.3.1.

To better understand the safety, tolerability and PK of INC280 and cetuximab combination, additional cohorts of patients may be enrolled at preceding dose levels, or to intermediate dose levels before or while proceeding with further dose escalation.

If a decision is made to escalate to a higher dose level but one or more additional patient(s) treated at the preceding dose level experiences a DLT during the first cycle of treatment, then the BLRM will be updated with this new information before any additional patients are enrolled at that higher dose level. Subjects ongoing will continue treatment at their assigned dose levels.

Implementation of Dose Escalation Decisions:

To Implement dose escalation decisions, the available toxicity information (including adverse events and laboratory abnormalities that are not DLTs), the recommendations from the BLRM, and the available PK and PD information will all be evaluated by the Investigators and Novartis study personnel (including the study physician and statistician) during a dose decision meeting by teleconference. Drug administration at the next higher dose level may not proceed until the Investigator receives written confirmation from Novartis indicating that the results of the previous dose level were evaluated and that it Is permissible to proceed to a higher dose level.

Intra-Patient Dose Escalation:

All patients in the dose escalation part will move to the MTD/RDE dose level once it is defined if it is considered appropriate in the opinion of the Investigator and Novartis. In order for a patient to be escalated to the MTD/RDE dose level, he or she must have tolerated their current dose for at least 4 cycles of therapy (i.e. he or she must not have experienced any INC280-related toxicity CTCAE grade a 2 at the dose level originally assigned). Consultation and agreement with Novartis must occur prior to any patient escalation at the MTD/RDE dose level. These changes must be recorded on the Dosage Administration Record eCRF.

Definitions of Dose Limiting Toxicities

A DLT is defined as an adverse event or abnormal laboratory value assessed as unrelated to disease, disease progression, inter-current illness, or concomitant medications that occurs within the first 28 days (first cycle) of treatment with INC280 In combination with cetuximab and meets any of the criteria Included in Table 4. National Cancer Institute CTCAE version 4.03 will be used for all grading. For the purpose of dose-escalation decisions, DLTs will be considered and Included in the BLRM.

The investigator must notify Novartis immediately of any unexpected CTCAE grade ≥3 adverse events or laboratory abnormalities. Prior to enrolling patients into a higher dose level, CTCAE grade ≥2 adverse events will be reviewed for all patients at the current dose level.

Note: Infusion-related reactions are not considered DLTs. Patients experiencing a severe infusion-related reaction during cycle 1 should be discontinued from the study

TABLE 4
Criteria for defining dose-limiting toxicities

| TOXICITY | DLT CRITERIA (Grade as in CTCAE version 4.03) |
|---|---|
| Blood and lymphatic system | Any Grade 4 of any duration |
| disorders^{a} | Febrile neutropenia (ANC <1.0 × 10⁹/L or 1000/mm³ and a single temperature of |
| | >38.3° C. (101 ° F.) or a sustained temperature of ≥38° C. (100.4° F.) for more than one |
| | hour) |
| General disorders and | Fatigue Grade 3 for >7 consecutive days |
| administration site conditions | Fatigue Grade 4 |
| Skin and subcutaneous tissue | Rash or photosensitivity Grade 3 for ≥14 consecutive days despite skin toxicity |
| disorders: | treatment (as per local practice and/or international guidelines) |
| Rash and/or photosensitivity | Rash or photosensitivity Grade 4 |
| Gastrointestinal disorders | Diarrhea Grade 3 for >48 hrs, despite the use of optimal anti-diarrhea therapy |
| | Diarrhea Grade 4 |
| | Nausea/vomiting Grade 3 for >48 hrs and Grade 4, despite the use of optimal anti- |
| | emetic therapy |
| Investigations (Metabolic) | Serum lipase or serum amylase (asymptomatic) Grade 3 >7 consecutive days |
| | Serum lipase or serum amylase (asymptomatic) Grade 4 |
| | Symptomatic elevation of serum lipase or serum amylase of any grade that requires |
| | medical intervention |
| Investigations (Renal) | Serum creatinine Grade ≥3 |
| Investigations (Hepatic)^{b} | Blood bilirubin (total bilirubin) Grade ≥3 |
| | AST or ALT Grade 3 for >7 consecutive days |
| | AST or ALT Grade 4 |
| Cardiac disorders | Any Grade ≥3 |
| Neurologic disorders | Any neurological abnormality or toxicity Grade ≥2 |
| Metabolism and nutrition | Hypomagnesaemia Grade 4 for ≥7 consecutive days despite supplements |
| disorders | correction (as per local practice and/or international guidelines) or symptomatic |
| | Grade 3 |
| Other AEs^{c} | Any other Grade ≥3 toxicity |

^{a}Anemia Grade ≥3 will not be considered a DLT unless judged to be a hemolytic process secondary to study treatment. Lymphopenia Grade ≥3 will not be considered a DLT unless clinically significant
^{b}For any Grade 3 or 4 hepatic toxicity that does not resolve within 7 days to Grade ≤1 (or Grade ≤2 if liver infiltration with tumor present), an abdominal CT scan must be performed to assess if it is related to disease progression.
^{c}An AE must be clinically significant to be defined as a DLT. Study treatment-related fever and electrolyte abnormalities (including K, Na, Cl, Mg, HCO₃, Ca, PO₄) that are Grade 3 abnormalities will not be considered a DLT unless clinically significant. Patients may receive replacement therapy as per local institutional guidelines. Patients may receive supportive care (e.g. PRBCs) as per local institutional guidelines. G-CSF may be used to treat patients who have developed dose-limiting neutropenia, as per institutional guidelines, following discontinuation of INC280 and cetuximab treatment. Optimal therapy for vomiting or diarrhea will be based on institutional guidelines, with consideration of the prohibited medications listed in this protocol.

### Example 3: Combination of cMET Inhibitor INC280 and EGFR Inhibitor Panitumumab in Colon and Head & Neck Cancer Cell Lines

Similar to Example 1, combination of Panitumumab and INC280 were tested in CAL-33, CCK-81 and YD-38 cell lines in the absent or present of 75 ng/ml HGF.

YD-38, CAL-33 and CCK-81 cells were treated in 96-well format for 3 days with HGF alone, Panitumumab alone, Panitumumab as a signal agent and Panitumumab with INC280 in the present or absent of HGF. (FIGS. 6 and 7). Cell viability was measured using the CellTiter-Glo assay. % viability was plotted as bar graphs with mean values and standard deviations from triplicates. (FIG. 8). % inhibition data was displayed numerically as 8×8 dose grid. Each data point represents averaged data from 3 wells+standard deviation, and the color spectrum also represents the level of the inhibition.

1. A pharmaceutical combination comprising
(i) a MET tyrosine kinase inhibitor which is INC280 having the formula 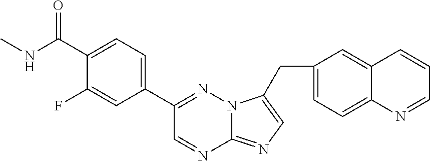 [SMILES: CNC(=O)c1ccc(-c2cnc3ncc(Cc4ccc5ncccc5c4)n3n2)cc1F]
or a pharmaceutically acceptable salt or hydrate thereof,
(ii) an EGFR tyrosine kinase Inhibitor which is a monoclonal antibody,
and optionally
(iiii) at least one pharmaceutically acceptable carrier.

2. The combination of claim 1, wherein the EGFR tyrosine kinase inhibitor is cetuximab.

3. The combination of claim 1, wherein the EGFR tyrosine kinase inhibitor is panitumumab.

4. The combination of claim 1, wherein the INC280 is in its dihydrochloric acid salt form.

5. The combination of claim 1, wherein the INC280 is a dihydrochloric monohydrate salt.

6. The combination of claim 1, wherein (i) and (ii) are simultaneously, separately or sequentially administered.

7. A method of treating an EGFR tyrosine kinase activity and/or MET tyrosine kinase activity mediated disease, especially a cancer, comprising administering a pharmaceutical combination comprising
(i) a MET tyrosine kinase inhibitor which is INC280 having the formula 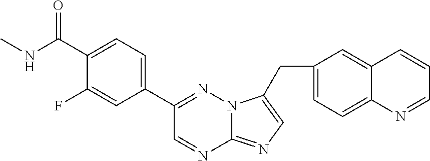 [SMILES: CNC(=O)c1ccc(-c2cnc3ncc(Cc4ccc5ncccc5c4)n3n2)cc1F]
or a pharmaceutically acceptable salt or hydrate thereof,
(ii) an EGFR tyrosine kinase inhibitor which is a monoclonal antibody,
and optionally
(iii) at least one pharmaceutically acceptable carrier.

8. The method of claim 7, wherein the EGFR tyrosine kinase Inhibitor is cetuximab.

9. The method of claim 7, wherein the EGFR tyrosine kinase inhibitor is panitumumab.

10. The method of claim 7, wherein the INC280 is in its dihydrochloric acid salt form.

11. The method of claim 7, wherein the INC280 is a dihydrochloric monohydrate salt.

12. The method of claim 7, wherein (i) and (ii) are simultaneously, separately or sequentially administered.

13. The method of claim 7, wherein the cancer is carcinomas (e.g., bladder, breast, cervical, cholangiocarcinoma, colorectal, esophageal, gastric, head and neck, kidney, liver, lung, nasopharygeal, ovarian, pancreas, prostate, thyroid); musculoskeletal sarcomas (e.g., osteosarcaoma, synovial sarcoma, rhabdomyosarcoma); soft tissue sarcomas (e.g., MFH/fibrosarcoma, leiomyosarcoma, Kaposi's sarcoma); hematopoietic malignancies (e.g., multiple myeloma, lymphomas, adult T cell leukemia, acute myelogenous leukemia, chronic myeloid leukemia); and other neoplasms (e.g., glioblastomas, astrocytomas, melanoma, mesothelioma and Wilm's tumor.

14. The method of claim 7, wherein the cancer is non-small cell lung cancer (NSCLC).

15. The method of claim 7, wherein the cancer is metastatic non-small cell lung cancer.

16. The method of claim 7, wherein the cancer is colorectal cancer (CRC).

17. The method of claim 7, wherein the cancer is metastatic colorectal cancer (mCRC).

18. The method of claim 7, wherein the cancer is head and neck cancer.

19. The method of claim 7, wherein the cancer is metastatic head and neck cancer.

20. The method of claim 7, wherein the cancer is head and neck squamous cell carcinoma (HNSCC).

21. The method of claim 7, wherein the cancer is mCRC in patients whose tumors have become resistant to anti-EGFR treatment through activation of the MET receptor.

22. The method of claim 7, wherein the cancer is HNSCC in patients whose tumors have become resistant to anti-EGFR treatment through activation of the MET receptor.
